SEQUENCE LISTING (4 sequences)

SEQ ID NO: 1 - DNA, 5217 bp, Mus musculus
  CDS (62)..(5128)
ttcctagctctgcaaaggacaggcctcgcgcaggatcccggcggacttctgaggtgccacgatggtaggggagctacgatacagggaattcagggtgcccctggggcctggcttgcacgcgtatccggatgaattgatccgccaacgggttggccataatgggcaccccgagtatcagatccgctggctcatcctcaggcgcggggatgatggggaccgggactctacagtggactgcaaggctgagcatatcctgttatggatgtctgacgatgagatctatgccaactgccacaagatgctgggcgagaatggccaagtcatcgcaccttcccgggagtccactgaggcaggggccctcgacaagtctgtgctgggggagatggaaacagatgtgaagtccttgattcagagggcccttcggcagctggaggagtgcgtgggcaccgtgcctcctgcccctctccttcacacggtccatgtactcagtgcctatgccagcatcgagcccctcactggcatcttcaaagaccgcagggttgtgaacttgctcatgcacatgttgagcagtcctgattatcagatccgctggagcgcaggccggatgatccaagctctgtcctcccacgatgctgggacccggacccagatccttctgtcattgagccaacaagaggccattgaaaagcacctggattttgatagccgctgcgctctgcttgcactgttcgcccaggctactctcacggaacacccgatgtctttcgagggcgttcagctgccacaggtcccaggacggctgctcttctccctggtaaaacgctacctgcacgtcaccttcctcctggatcggctgaacggcgatgcaggggatcaaggagcccagaacaactttagtcctgaggagttgaatgtagggaggggccggctggaactggaattcagtatggccatgggcactctgatctctgagctggtgcaggccatgcgctgggacggggcctcaagcagaccagagagttcttcctcctccaccttccagcctcggccagcacagttccgcccctacacccagcgtttcaggaggtcgaggcggtttcgcccccgtgcctcgtttgccagttttaatacctatgccttgtatgtgcgggacacgctgcggcccgggatgcgggtacggatgctggagaattacgaggagatcgctgctggggatgagggccagttccgacagagcaatgatggcgttcccccagcgcaggtgttgtgggattcaacaggccatacctactgggtgcactggcacatgctggagatcttgggctttgaggaagacatcgaggatgtgattgatattgaagagttacaggagctaggggccaatggagcactgagcatcgtcccgccgtcccagcgctggaagcccataactcagctctttgccgagccttacgtggtacccgaggaggaagacagggaagagagcgagaacttgacccaggctgagtggtgggagctcctcttcttcatccggcagttgagtgaggcagagcgccttcacatcgtggatctcctgcaagaccacctggaagaggagcgcgttctggactacgatatgctgcctgagctgaccgtgcccgttgacttggcccaggatctgctgttgtctctgcctcagcaacttgaggacagtgctctgagggacctgttcagctgcagtgtctacaggaagtatgggcccgaagtcctggtagggcatctaagctacccatttgtgccaggtgcccagccaaatttattcggagccaatgaagagtctgaagccaaagatcccccacttcagagtgccagccctgccctgcagcgcctggtggagagcttgggccccgaaggggaggtccttgtggaactggaacaagccctcggctccgaggctccccaggaaactgaggtcaagtcctgcttgttgcagctccaggagcagccccagcccttcctcgctctgatgcggagcctggacacttccgccagcaacaagaccctgcacctcactgtgctcagaatcttaatgcagctggtgaacttcccagaggcgctgttgctaccctggcacgaggccatggatgcctgcgtgacctgccttcggtcccccaatactgaccgagaggtgctccaggaactaatctttttcctgcaccgcctgaccaccacaagccgggactatgcggtgatactaaaccagcatggagcccgggacgccatctccaaagtcctggaaaagcaccgagggaaactggagttggctcaggagctgcgggatatggtgtccaagtgtgagaagcatgcccacctctaccggaaactcaccaccaacatcctgggcggttgcatccagatggtcctgggtcagattgaagaccacagacgaacccaccggcccatccaaatcccattctttgatgtgtttctcagatatctgtgccagggctccagtgaggaaatgaagaaaaacaggtactgggagaaggtggaggtgtcctccaacccacagcgggccagcaggctgacggaccgcaaccccaagacctactgggagtccagtggcagggccggctcccacttcatcaccttacacatgcgcccaggtgtcatcatcaggcagctgactctactggtggctggcgaggactcaagctacatgccagcctgggtggtggtatgcgggggcaacagcatcaagtccgttaataaagaactcaacacggtaaacgtgatgccctctgccagccgggtgaccctcctggagaacctgacccgcttctggcccatcatccaaatcagaataaagcgctgccagcagggtggcattaacacgcgcatccggggcctagaggtgctgggccccaagcccaccttctggccagtgttccgagagcaactgtgccggcacacgcgcctcttctacatggttcgggcccaggcatggagtcaggacatagcagaggaccgccggagccttctgcacctgagttctaggctaaatggggctctgcgccatgaacagaattttgcagagcgcttccttcctgatatggaggccgcccaagcactgagcaagacctgctgggaggcgctggtcagccccctggtgcagaacattacatctcccgatgaggacagcaccagctccttgggctggctgctggatcagtacttgggatgcagggaggctgcctacaatccccagagcagggctgctgctttctcctcccgggttcgccgccttacccacctcctggtccatgtggagccccgtgaggcagcacctccggtggtggccatccctcgatccaagggcaggaacagaatccatgactggagctacttgatcacccggggccttccaagtagcatcatgaagaacctgacccgctgttggcggtctgtggtggaggagcagatgaacaagtttctcagtgcgtcctggaaagacgatgatttcgtaccccgctattgcgagcgctattacgtcctgcagaagtccagctcagagctgtttgggccacgagctgccttcttgctggcaatgcggaatggctgtgctgatgctgtgcggaggctccctttcctcagggccgcccatgtgaagcagcagtttgctcggcacattgaccagaggatccaaggcagtaggatgggtggagcccggggaatggagatgctggcacagttgcagcgatgcctggagtctgtcctgattttctctcccctggagatagccaccacctttgagcattactaccagcactacatggctgaccgtctcctgagcgtaggctccagctggctggagggggccgtactggagcagatcggtccctgcttccccagccgtcttccccagcagatgctacagagcctgaacgtctcagaggagttgcagcgtcagttccacgtttaccagctgcagcagcttgatcaggagctcctgaagctggaagacacggaaaagaagatacaggtggcccatgaggacagtggcagagaggacaagagcaagaaggaagaagccattggagaagccgcggctgtggctatggcagaggaggaggatcaagggaagaaggaggagggagaggaggaaggggagggagaggatgaggaggaagagcgctattataaaggaacaatgccagaagtgtgtgtacttgtcgtgacgccacgcttctggcctgtcgcctccgtctgccaaatgctcaacccggcaacgtgcctgcccgcatacctgcgggggaccataaaccactacaccaacttttacagcaagagtcagagccgctccagcttagagaaagagccacagaggcggctgcagtggacctggcagggccgggcagaagtgcagttcgggggtcagattctgcatgtgtccacagtacagatgtggctgctgctgcatctcaacaaccaaaaggaggtgtctgtcgagagcctgcaggctatctcggagctccctccagatgtgcttcacagggccatcgggcctctcacctcatcaagaggtcccttggacctgcaggagcagaagaacgtaccaggaggggtgctcaagattcgagatgacagtgaggaacccaggccgaggaggggcaacgtgtggctgatcccacctcagacatacctacaagctgaggccgaagagggccggaacatggagaagagaaggaatcttctgaattgccttgttgtccgaatcctcaaggctcacggggatgaaggcttgcatgttgaccggctcgtctatctggtgctagaagcgtgggagaaaggcccgtgtcctgctaggggtctggtcagcagcctcggcaggggagcaacctgcaggagctctgatgtcctctcctgcatcctgcacctcctggtcaagggcacgctgagacgccatgacgaccggccgcaggtgctgtactatgcagttcctgtaactgtgatggagccccacatggagtccctgaaccctggctcggcaggccccaatccacccctcaccttccacaccctgcagattcgatcccggggtgtgccttacgcctcctgcactgataaccacaccttctccactttccggtagccctggatatggggttgggggtaggtgaggctggggctttcatcagaaaataaaatgctggaatttgaaaaaaaaaaaaaaaaaaa

SEQ ID NO: 2 - protein, 1689 aa, Mus musculus
MVGELRYREFRVPLGPGLHAYPDELIRQRVGHNGHPEYQIRWLILRRGDDGDRDSTVDCKAEHILLWMSDDEIYANCHKMLGENGQVIAPSRESTEAGALDKSVLGEMETDVKSLIQRALRQLEECVGTVPPAPLLHTVHVLSAYASIEPLTGIFKDRRVVNLLMHMLSSPDYQIRWSAGRMIQALSSHDAGTRTQILLSLSQQEAIEKHLDFDSRCALLALFAQATLTEHPMSFEGVQLPQVPGRLLFSLVKRYLHVTFLLDRLNGDAGDQGAQNNFSPEELNVGRGRLELEFSMAMGTLISELVQAMRWDGASSRPESSSSSTFQPRPAQFRPYTQRFRRSRRFRPRASFASFNTYALYVRDTLRPGMRVRMLENYEEIAAGDEGQFRQSNDGVPPAQVLWDSTGHTYWVHWHMLEILGFEEDIEDVIDIEELQELGANGALSIVPPSQRWKPITQLFAEPYVVPEEEDREESENLTQAEWWELLFFIRQLSEAERLHIVDLLQDHLEEERVLDYDMLPELTVPVDLAQDLLLSLPQQLEDSALRDLFSCSVYRKYGPEVLVGHLSYPFVPGAQPNLFGANEESEAKDPPLQSASPALQRLVESLGPEGEVLVELEQALGSEAPQETEVKSCLLQLQEQPQPFLALMRSLDTSASNKTLHLTVLRILMQLVNFPEALLLPWHEAMDACVTCLRSPNTDREVLQELIFFLHRLTTTSRDYAVILNQHGARDAISKVLEKHRGKLELAQELRDMVSKCEKHAHLYRKLTTNILGGCIQMVLGQIEDHRRTHRPIQIPFFDVFLRYLCQGSSEEMKKNRYWEKVEVSSNPQRASRLTDRNPKTYWESSGRAGSHFITLHMRPGVIIRQLTLLVAGEDSSYMPAWVVVCGGNSIKSVNKELNTVNVMPSASRVTLLENLTRFWPIIQIRIKRCQQGGINTRIRGLEVLGPKPTFWPVFREQLCRHTRLFYMVRAQAWSQDIAEDRRSLLHLSSRLNGALRHEQNFAERFLPDMEAAQALSKTCWEALVSPLVQNITSPDEDSTSSLGWLLDQYLGCREAAYNPQSRAAAFSSRVRRLTHLLVHVEPREAAPPVVAIPRSKGRNRIHDWSYLITRGLPSSIMKNLTRCWRSVVEEQMNKFLSASWKDDDFVPRYCERYYVLQKSSSELFGPRAAFLLAMRNGCADAVRRLPFLRAAHVKQQFARHIDQRIQGSRMGGARGMEMLAQLQRCLESVLIFSPLEIATTFEHYYQHYMADRLLSVGSSWLEGAVLEQIGPCFPSRLPQQMLQSLNVSEELQRQFHVYQLQQLDQELLKLEDTEKKIQVAHEDSGREDKSKKEEAIGEAAAVAMAEEEDQGKKEEGEEEGEGEDEEEERYYKGTMPEVCVLVVTPRFWPVASVCQMLNPATCLPAYLRGTINHYTNFYSKSQSRSSLEKEPQRRLQWTWQGRAEVQFGGQILHVSTVQMWLLLHLNNQKEVSVESLQAISELPPDVLHRAIGPLTSSRGPLDLQEQKNVPGGVLKIRDDSEEPRPRRGNVWLIPPQTYLQAEAEEGRNMEKRRNLLNCLVVRILKAHGDEGLHVDRLVYLVLEAWEKGPCPARGLVSSLGRGATCRSSDVLSCILHLLVKGTLRRHDDRPQVLYYAVPVTVMEPHMESLNPGSAGPNPPLTFHTLQIRSRGVPYASCTDNHTFSTFR

SEQ ID NO: 3 - DNA, 5253 bp, Homo sapiens
  CDS (87)..(5183)
gtcgccgccagcgtctgtgccgcgtcccttgctctgtgaaggacaggcctcgcgccaggaccccggtggacttctgaggtgccaggatggtgggagaactccgctacagggaattcagggtgcccctggggcccggcttacatgcctatcctgatgagctgatccgccagcgcgtgggccatgatgggcatcctgagtaccagatccgttggctcatcctgcggcgtggcgatgagggggacgggggctctggccaagtggactgcaaggctgagcacatcctgctgtggatgtccaaggatgagatctatgccaactgccacaagatgctgggcgaggatggccaggtcatcgggccctcccaggagtctgcaggggaggttggggccctggacaaatctgtgctggaggagatggaaaccgatgtgaagtccctcattcagagagcccttcggcagctggaggagtgtgtgggcactatccctcctgctcctctacttcacactgtccacgtgctcagcgcctatgccagcattgagcccctcactggagtattcaaggacccaagggtcctggacttgctcatgcacatgttgagtagtcctgattatcagattcgctggagtgcaggccggatgatacaagccctgtcctcccatgacgctgggacccggactcagatccttctgtcactgagccaacaagaagccattgagaaacacctggattttgacagccgctgtgctctgctagcactgtttgcacaggccacgctctctgaacaccccatgtctttcgagggcattcagctaccacaggtcccaggaagggtgctcttctccctggtgaagcggtatttgcatgtcacctcgctcctggatcagctgaacgacagtgctgcggagccaggagcccagaacacctctgctcctgaggagttgagtggggagaggggtcaactggagctggagttcagtatggccatgggcaccctgatctcggagctggtgcaagccatgcgctgggaccaggcctcagacagaccaaggagctcagcacggtcccccggttccatcttccagcctcagctggcagatgtgagcccagggctccccgctgcccaggctcagccctccttcaggaggtcaagacgttttcgccctcgttctgagttcgcaagtggcaatacctatgctttgtatgtgcgggacacactgcagccggggatgcgagtgcggatgctggatgattatgaggagatcagtgccggggatgagggcgagtttcggcagagcaacaacggtgtgcctcctgtgcaggtattttgggagtcaacaggccgcacctattgggtgcactggcacatgctggagatcttgggctttgaggaagacattgaggacatggttgaggctgatgagtaccaaggggcagtggccagtagagtcctgggtagagccctgcctgcctggcgctggaggcccatgacagaactctatgctgtgccttatgtgctgcctgaggatgaggacactgaggagtgtgaacacctgaccctggctgagtggtgggaactcctcttcttcatcaagaagctggatggacctgaccatcaggaggttctccagatcctccaggagaacctagatggggagattctggatgatgagatcctagctgaactggccgtgcccatagaattggcccaggacttgctgctgactctgccacagcgactcaatgacagtgccctcagggacctgatcaactgccatgtctacaagaagtatgggcctgaagccctagcagggaaccaagcctacccatcccttctagaagcccaagaagatgtcctcctgctagacgcgcaggcccaggctaaggactcagaagatgcagccaaagtggaagcaaaagaacccccatctcagagtcccaacactcccctgcagcgtctggtggagggttatggtccagctgggaaaatcctcctggatctagagcaagccctcagctcagaggggacccaggagaacaaggtcaagccactcctgctgcagctgcagcggcagccgcagcccttcctggcactgatgcagagcctggacactccggagactaacaggaccctgcacctgactgtgctgagaatcctgaagcagctggtggacttccccgaggcactgctgctcccctggcacgaggccgtggatgcctgcatggcctgcctgcggtccccaaacactgatcgagaggtgctccaggaactgattttcttcctgcaccgcctgacctcagtgagcagggactatgccgtggtgctgaatcagctgggagcaagagacgctatctccaaggccctggaaaagcacctgggaaagctggagctggctcaggagctgcgggacatggtgttcaagtgtgagaagcatgcccacctctaccgcaaactcatcaccaacatcctgggaggctgcatccagatggtgctgggccagatcgaagaccacagacgaacccaccggcccatcaacatccctttctttgatgtgttcctcagatacctgtgccagggctccagtgtggaagtgaaggaggacaagtgctgggagaaggtggaggtgtcctccaacccgcaccgggccagcaagctgacggaccacaaccccaagacctattgggagtccaacggcagcgccggctcccactacatcaccctgcacatgcgccggggcatcctcatcaggcaactgactctgcttgtggctagtgaggactcgagttacatgccggcccgagtggtggtgtgcgggggtgatagcactagctctcttcacacggaactcaactcggtgaatgtgatgccctctgccagccgggtgatcctcctggagaacctgacccgcttctggcccatcatccagatccgcataaagcgctgccagcagggtggcattgatacgcgcattcgggggttagagatcctaggccccaagcccacgttctggccagtgttccgggagcagctctgtcgtcacacacgcctcttctacatggttcgggcacaggcctggagccaggacatggcagaggaccgcaggagcctcctgcacctgagttctagactcaacggtgctctgcgccaggagcagaattttgctgaccgcttcctccctgatgacgaggctgcccaagctctgggcaagacctgctgggaggccctggtcagccccgtggtgcagaacatcacctcccctgatgaggatggcattagccccctgggttggctgctggaccagtacctggagtgtcaggaagctgtcttcaacccccagagccgcggcccagctttcttctcgcgggtgcgccgtctcactcacctgctggtgcatgtcgagccctgtgaggcaccccctcctgtggtggccactcctcggcccaaaggcagaaacagaagccacgactggagctccttggctacccggggccttccaagcagcatcatgagaaacctgacgcgctgttggcgggccgtggtggagaagcaggtgaacaattttctgacctcatcctggcgggatgatgactttgtgccacgctactgtgagcactttaatattctgcagaactcaagctctgaactgtttgggcctcgggcagccttcttgctggcgctgcaaaatggctgtgcgggagccttgctgaagctcccttttctcaaagctgcccacgtgagtgagcagttcgcccggcacattgaccagcagatccagggcagccggatcggtggagcccaggaaatggagaggctggcacagctgcagcaatgcctgcaagctgtcctgattttctccggcttggagatagccaccacttttgagcattattaccagcactacatggcggaccgtctcctgggcgtggtctcgagctggctggagggggccgtgctggagcagatcggtccctgcttccccaaccgcctcccccagcagatgttgcagagcctgagcacctctaaggagctgcagcgccagttccacgtctaccagctccagcagctggatcaggaactcctaaagctggaggatacagagaagaaaatacaggtgggccttggggccagtggcaaggagcacaagagcgagaaggaagaggaagctggggcagcagcagtggtggatgtggcggagggagaggaggaagaggaggagaatgaggacctctactatgaaggggcaatgccagaagtgtctgtgcttgtcctgtcccgacactcctggcctgttgcctcaatctgccacacactgaaccccagaacctgcctgccctcctacctgaggggcactttgaacagatactccaacttctacaacaagagtcagagccaccctgcccttgagcgaggctcacagaggcgactgcagtggacgtggctgggctgggctgagctgcagtttgggaaccagaccctgcatgtgtccaccgtgcagatgtggctactgctgtatctcaacgacctgaaggcggtctctgtggagagtctgctggcgttctcagggctctccgcagacatgctcaatcaggcgattgggcccctcacctcttcaagaggccccctggaccttcacgagcaaaaggatataccaggaggggtcctcaagattcgagatggcagcaaggaacccaggtcgagatgggacattgtgcggctcatcccacctcagacgtacctgcaagctgagggtgaagacggccagaacttggagaagagacggaatcttctgaactgcctcatcgtccgaatcctcaaggcccatggagatgaggggctgcacattgaccagcttgtctgtctggtgctggaggcttggcagaagggcccgtgtcctcccaggggtttggtcagcagccttggtaaggggtctgcatgcagcagcactgacgtcctctcctgcatcctacacctcctgggcaagggcacgctgagacgccatgacgaccggccccaggtgctgtcctatgcagtccctgtgactgtcatggagcctcacactgagtccctgaacccaggctcctcaggccccaacccacccctcaccttccataccctacagattcgctcccggggtgtgccctatgcctcctgcactgccacccagagcttctctaccttccggtagccctagacttggggtcaggggaaggtagagctggagcttttacagaaattaaacccaagagtttgattat

SEQ ID NO: 4 - protein, 1698 aa, Homo sapiens
MVGELRYREFRVPLGPGLHAYPDELIRQRVGHDGHPEYQIRWLILRRGDEGDGGSGQVDCKAEHILLWMSKDEIYANCHKMLGEDGQVIGPSQESAGEVGALDKSVLEEMETDVKSLIQRALRQLEECVGTIPPAPLLHTVHVLSAYASIEPLTGVFKDPRVLDLLMHMLSSPDYQIRWSAGRMIQALSSHDAGTRTQILLSLSQQEAIEKHLDFDSRCALLALFAQATLSEHPMSFEGIQLPQVPGRVLFSLVKRYLHVTSLLDQLNDSAAEPGAQNTSAPEELSGERGQLELEFSMAMGTLISELVQAMRWDQASDRPRSSARSPGSIFQPQLADVSPGLPAAQAQPSFRRSRRFRPRSEFASGNTYALYVRDTLQPGMRVRMLDDYEEISAGDEGEFRQSNNGVPPVQVFWESTGRTYWVHWHMLEILGFEEDIEDMVEADEYQGAVASRVLGRALPAWRWRPMTELYAVPYVLPEDEDTEECEHLTLAEWWELLFFIKKLDGPDHQEVLQILQENLDGEILDDEILAELAVPIELAQDLLLTLPQRLNDSALRDLINCHVYKKYGPEALAGNQAYPSLLEAQEDVLLLDAQAQAKDSEDAAKVEAKEPPSQSPNTPLQRLVEGYGPAGKILLDLEQALSSEGTQENKVKPLLLQLQRQPQPFLALMQSLDTPETNRTLHLTVLRILKQLVDFPEALLLPWHEAVDACMACLRSPNTDREVLQELIFFLHRLTSVSRDYAVVLNQLGARDAISKALEKHLGKLELAQELRDMVFKCEKHAHLYRKLITNILGGCIQMVLGQIEDHRRTHRPINIPFFDVFLRYLCQGSSVEVKEDKCWEKVEVSSNPHRASKLTDHNPKTYWESNGSAGSHYITLHMRRGILIRQLTLLVASEDSSYMPARVVVCGGDSTSSLHTELNSVNVMPSASRVILLENLTRFWPIIQIRIKRCQQGGIDTRIRGLEILGPKPTFWPVFREQLCRHTRLFYMVRAQAWSQDMAEDRRSLLHLSSRLNGALRQEQNFADRFLPDDEAAQALGKTCWEALVSPVVQNITSPDEDGISPLGWLLDQYLECQEAVFNPQSRGPAFFSRVRRLTHLLVHVEPCEAPPPVVATPRPKGRNRSHDWSSLATRGLPSSIMRNLTRCWRAVVEKQVNNFLTSSWRDDDFVPRYCEHFNILQNSSSELFGPRAAFLLALQNGCAGALLKLPFLKAAHVSEQFARHIDQQIQGSRIGGAQEMERLAQLQQCLQAVLIFSGLEIATTFEHYYQHYMADRLLGVVSSWLEGAVLEQIGPCFPNRLPQQMLQSLSTSKELQRQFHVYQLQQLDQELLKLEDTEKKIQVGLGASGKEHKSEKEEEAGAAAVVDVAEGEEEEEENEDLYYEGAMPEVSVLVLSRHSWPVASICHTLNPRTCLPSYLRGTLNRYSNFYNKSQSHPALERGSQRRLQWTWLGWAELQFGNQTLHVSTVQMWLLLYLNDLKAVSVESLLAFSGLSADMLNQAIGPLTSSRGPLDLHEQKDIPGGVLKIRDGSKEPRSRWDIVRLIPPQTYLQAEGEDGQNLEKRRNLLNCLIVRILKAHGDEGLHIDQLVCLVLEAWQKGPCPPRGLVSSLGKGSACSSTDVLSCILHLLGKGTLRRHDDRPQVLSYAVPVTVMEPHTESLNPGSSGPNPPLTFHTLQIRSRGVPYASCTATQSFSTFR

(19) United States
(12) Patent Application Publication
(10) Pub. No.: US 2003/0073236 A1
(43) Pub. Date: Apr. 17, 2003

(54) p193 proteins and nucleic acids, and uses thereof

(76) Inventors: Loren J. Field, Indianapolis, IN (US); Shih-Chong Tsai, Taipei (TW)

Correspondence Address: Kenneth A. Gandy, Woodard, Emhardt, Naughton, Moriarty & McNett, Bank One Center/Tower, 111 Monument Circle, Suite 3700, Indianapolis, IN, 46204-5137, US

(21) Appl. No.: 10/080,943

(22) Filed: Feb. 22, 2002

Related U.S. Application Data
(63) Continuation of application No. PCT/US00/23161, filed on Aug. 23, 2000.
(60) Provisional application No. 60/150,266, filed on Aug. 23, 1999.

(51) Int. Cl. A61K 48/00; C12N 15/85

(52) U.S. Cl. 435/455; 514/44

(57) ABSTRACT

Described are nucleotide sequences coding for and regulating expression of a cell cycle regulatory protein, designated p193, and recombinant vectors and host cells containing nucleotide sequences coding for and regulating expression of the protein sequence. Also described are methods for modifying the cell cycle of a cell by regulating p193 or its pathway. Methods for inducing apoptosis in cells are provided by increasing level of a pro-apoptotic p193 protein in the cells. Methods for suppressing apoptosis in or increasing the proliferative potential of cells are provided by reducing the level of pro-apoptotic p193 protein the cells and/or by interfering with the native p193 signal transduction pathway, for instance utilizing a p193 protein having a dominant negative mutation.

### REFERENCE TO RELATED APPLICATION

This application claims the benefit of U.S. patent application Ser. No. 60/150,266 filed Aug. 23, 2000, which is hereby incorporated by reference in its entirety.

### BACKGROUND

The present invention relates generally to cell physiology, and more particularly to cell cycle regulatory proteins. Specifically, the present invention relates to a novel apoptosis associated protein designated p193 and modified forms thereof; to nucleotide sequences encoding p193 proteins; and to products and processes involved in the cloning, preparation and expression of nucleotide sequences encoding p193.

Normal development is dependent upon an intricate balance between cell proliferation and programmed cell death (apoptosis). Alteration of this balance can have significant pathophysiological consequences; tumorigenesis results when cell proliferation is favored whereas autoimmune and/or degenerative disorders result when apoptosis is favored.

In mammalian cells, apoptosis can be induced by at least two independent regulatory pathways. The first pathway relies on direct activation of the death receptors (members of the tumor necrosis factor receptor superfamily, reviewed in Ashkenazi, A. et al. (1998) Science 281, 1305-1308). For example, activation of the TNFR1 or CD95 receptors initiates a signal transduction cascade primarily through FADD (Fas-associated death domain) which rapidly activates caspase 8, thereby initiating apoptosis. Apoptosis can also be regulated through the activities of Bcl-2 family members (reviewed in Adams, J. M. et al. (1998) Science 281, 1322-1326). The prototypical family member, Bcl-2, was originally identified as a gene activated by chromosomal translocation in some human lymphomas (Tsujimoto, Y. et al. (1984) Science 226, 1097-1099; Bakhshi, A. et al. (1985), Cell 41, 899-906; Cleary, M. L. et al. (1986) Cell 47, 19-28). Subsequent analyses have identified a family of approximately 20 proteins which share homology to Bcl-2 at one or more domains (known as Bcl-2 Homology domains BH1 through BH4). Functional analyses have shown that family members with the greatest homology to Bcl-2 tend to promote cell survival while those more distantly related tend to promote apoptosis. The pro-apoptosis group is further subdivided into the Bax sub-family (which contain BH1, 2 and 3 domains, see Oltvai, Z. N. et al. (1993) Cell 74, 609-619; Chittenden, T. et al. (1995) Nature 374, 733-736; Kiefer, M. C., et al. (1995) Nature 374, 736-739; Farrow, S. N. et al. (1995) Nature 374, 731-733; Hsu, Y. T. et al. (1997) Proc. Natl. Acad. Sci. USA 94, 3668-3672) and the BH3 only sub-family (which as the name implies contain only BH3 domains, see Boyd, J. M. et al. (1994) Cell 79, 341-351; Boyd, J. M., et al., (1995) Oncogene 11, 1921-1928; Yang, E. et al., (1995) Cell 80, 285-291; Wang, K. et al. (1996) Genes Dev. 10, 2859-2869; Inohara, N. et al. (1997) EMBO J. 16, 1686-1694; Conradt, B. et al. (1998) Cell 93, 519-529; O'Connor, L. et al. (1998) EMBO J. 17, 384-395, Hegde, R. et al. (1998) Journal of Biological, Chemistry 273, 7783-7786. 11-18).

Commitment to apoptosis is governed, at least in part, by the relative levels of pro-survival and pro-apoptosis Bcl-2 family members which, in turn, regulate the activity of Apaf-1 (an activator of caspase 8). Thus the caspase family of cysteine proteases are the downstream effectors of apoptosis, regardless of the initial regulatory pathway. Once activated, the caspases effect cell death by initiating a proteolytic cascade which destroys cellular organelles thereby giving rise to distinct morphologic changes which are diagnostic for apoptosis (reviewed in Thornberry, N. A. et al. (1998) Science 281, 1312-1316). These include nuclear condensation, fragmentation of DNA at nucleosomal junctions, mitochondrial disintegration and ultimately autolysis of the cell.

The DNA tumor virus oncoproteins have provided a more useful model system with which to dissect the molecular regulation of cell growth and death. The transforming activities of these proteins (as exemplified by SV40 Large T Antigen and Adenovirus E1A) reside largely in their ability to bind to, and thereby alter the activity of, endogenous cell cycle and cell death regulatory proteins (reviewed in Ludlow, J. W. et al. (1995) Virus Research 35, 113-121; and Moran, E. (1993) FASEB Journal 7, 880-885). In the case of T Antigen (T-Ag), amino acid residues 105 through 115 are required for binding to members of the Retinoblastoma family (RB and the related proteins p107 and p130, see DeCaprio, J. A. et al. (1988) Cell 54, 275-283; Ewen, M. E. et al. (1991) Cell 66, 1155-1164; Li, Y. et al. (1993) Genes Dev. 7, 2366-2377; and Hannon, G. J. et al. (1993) Genes Dev. Dec. 7, 2378-2391). T-Ag/RB binding blocks sequestration of E2F family members (which are maintained in an inactive state by binding to RB). Once released, these transcription factors activate expression of a large number of genes needed for S phase entry (reviewed in Nevins, J. R. (1992) Science 258, 424-429; Hatakeyama, M. et al. (1995) Prog. Cell cycle Res. 1, 9-19; and La Thangue, N. B. (1996) Bioch. Soc. Trans. 24, 54-59). The discontinuous region localized between T-Ag amino acid residues 350 through 450 and 532 through 625 is required for binding to p53 (Kierstead, T. D. et al. (1993) J. Virol. 67, 1817-1829). Among other activities, p53 functions as a transcriptional co-activator of both pro-apoptosis and growth inhibitory genes. T-Ag/p53 binding prevents transcriptional activation of these genes, and concomitantly inhibits their activities (Bates, S. et al. (1999) Cell. & Mol. Life Sci. 55, 28-37; and Ko, L. J. et al. (1996) Genes Dev. 10, 1054-1072).

### DESCRIPTION OF THE FIGURES

FIG. 1. (a) Immune complex from metabolically labeled AT-2 cardiomyocytes generated with anti-T-AG or anti-p53 monoclonal antibodies. p193 is present in anti-T-AG (lane 3) and anti-p53 (lanes 2 and 6) immune complex from ³⁵S-methionine labeled AT-2 cardiomyocytes, but not in immune complex prepared with IgG subtype-matched nonspecific control antibodies (lanes 1 and 5), nor in controls lacking primary antibody (lane 4). Molecular weight standards are indicated on the left. (b) PSD MALDI mass spectrum and sequence of a p193 tryptic peptide. The b and y ions and immonium ions that were detected are shown. (c) Schematic diagram of p193 protein and cDNAs. The positions of several structural motifs are shown. Horizontal black lines indicate the relative position of the cDNA clones.

FIG. 2. (a) Deduced amino acid sequence of p193. Underlined sequences correspond to the peptides identified by PSD mass spectrometry. Bold sequence corresponds to the BH3 domain homology. (b) Comparison of the BH3 domain in p193 and several other apoptosis regulatory proteins.

FIG. 3. (a) p193 binds to T-Ag in NIH-3T3 cells. Protein prepared from cells co-transfected with CMV-p193myc (which encodes a p193 protein harboring a c-terminal myc epitope tag) and CMV-T-Ag (which encodes SV40 T-Ag) was reacted with the indicated antibodies, and the resulting immune complex was analyzed by Western blotting using anti-myc and anti-T-Ag antibodies. Tfx, transfection; Tot. Pro., total protein; IP, immune precipitation. (b) In vitro translated p193 binds to recombinant T-Ag. Radiolabeled in vitro translated p193 was mixed with recombinant T-Ag, and then reacted with the indicated antibodies. The resulting immune complexes were displayed on a polyacrylamide gel and transferred to nylon membranes. p193 was visualized by autoradiography, and T-Ag was visualized by Western blot. (c) Northern blot analysis of p193 expression in adult mice. Total RNA (10 micrograms) prepared from the indicated tissues was probed with a full-length p193 cDNA. The integrity of the RNA samples was confirmed by staining the Northern blots with methylene blue (lower panel).

FIG. 4. p193 binds to the N-terminus of T-Ag. The schematic diagram depicts the T-Ag constructs used in the mapping experiments. These products were translated in vitro and mixed with in vitro translated full length p193. Immune complex generated with anti-T-Ag antibody PAb419 was resolved on a polyacrylamide gel and visualized by autoradiography. Construct 1-92myc encoded a myc epitope-tag at the C-terminus.

FIG. 5. p193 promotes apoptosis. (a) DNA content distribution for NIH-3T3 cells expression CMV-βGALmyc at 40 hrs post-transfection. (b) DNA content distribution for NIH-3T3 cells expressing CMV-p193myc at 40 hrs post-transfection. (c) Time course of cell death and DNA synthesis in synchronized cultures of NIH-3T3 cells transfected with CMV-p193myc. The % survival of CMV-p193myc expressing cells (squares), the thymidine labeling index for CVM-p193myc transfected cells (circles), and the thymidine labeling index for non-transfected NIH-3T3 cells on the same chamber slides (diamonds) are shown. (d and e) p193myc immune localization (signal corresponds to anti-myc epitope tag immune reactivity) in NIH-3T3 cells at 8 and 14 hrs, respectively, post serum replenishment. (f) Co-expression of Bcl-X_{L} or T-Ag antagonizes p193-induced apoptosis. NIH-3T3 cells were transfected as indicated; the total number of p193 positive cells at 68 hours post-transfection is shown. Also note that cells transfected with the p193deltaBH (which harbors a deletion spaning the p193 BH3 domain) are viable.

FIG. 6. (a and b) p193myc (panel a, signal corresponds to anti-p193myc immune reactivity) and T-Ag (panel b, signal corresponds to anti-T-Ag immune reactivity) are sequestered in the cytoplasm in cells co-expressing CMV-p193myc and CMV-T-Ag. (c) The percentage thymidine positive cells with cytoplasmic T-Ag immune reactivity is plotted against the number of hours post S-phase (as determined by pulse-chase experiments). (d and e) Autoradiographic and anti-T-Ag immune cytologic analysis, respectively, of two ³H-thymidine positive daughter cells after cytokinesis (from the 10 hrs chase time point in panel c).

FIG. 7. (A). NIH-3T3 colony growth assay with expression constructs encoding p193 in the sense (CMV-p193s) and anti-sense (CMV-p193as) orientation. Expression vector lacking insert (CMV-null) was used as a control. (B). RT-PCR analysis from cells expressing the CMV-null vector, from cells expressing the CMV-p193as vector, or from non-transfected NIH-3T3 cells.

FIG. 8. (A). Structure of CMV expression vectors with nested p193 C-terminal truncations, as described in Example 4. (B). Colony growth assay using expression constructs of FIG. 8A, as described in Example 4. (C) DNA fragmentation studies confirming that p193dn encodes dominant negative activity which blocks MMS-induced apoptosis, as described in Example 4.

FIG. 9. Schematic diagram of MHC-p193dn transgene used to generate transgenic mice, as further described in Example 5.

FIG. 10. Northern blot of transgene expression in MHC-p193dn transgenic mouse lines designated 4, 5, 6, 7, 9, 10 and 13, as further described in Example 5.

FIG. 11. Heart sections showing myocardial damage in response to isoproterenol infusion in control and MCH-p193dn transgenic mice, obtained as described in Example 6. Sections were stained with sirius red (which reacts with collagen to produce a dark signal) and counterstained with fast green (which reacts with cardiomyocytes to produce a light signal).

FIG. 12. ES cell-derived cardiomyocyte colony growth assay showing the effects of p53dn, p193dn, and E1A gene expression, alone or in combination, as further described in Example 7.

FIG. 13. (A). Western blot analysis of protein prepared from the ES cell-derived cardiomyocyte colony growth assay shown in FIG. 12 with anti-E1A or anti-T-Ag antibodies; (B). DNA fragmentation studies showing that E1A expression in the absence of co-expression of both p13dn and p193dn induced apoptosis (see Example 7).

FIG. 14. p193 is expressed in G₁/S of the cell cycle (see Example 8). (A) Plot of % tritiated thymidine postive cells over time showing that the NIH-3T3 culture studies were well synchronized (B). Western analysis of p193 expression over the same time period, as described in Example 8. The Western analyses indicate that p193 is expressed during G1/S.

FIG. 15. Colony growth assay demonstrating that isoproternol induces growth in cardiomyocytes which co-express 193dn and p53dn, as described in Example 9.

### SUMMARY OF THE INVENTION

A feature of the present invention is the identification and characterization of an apoptosis associated protein, designated p193. p193 is a SV40 T-Ag binding protein and appears to be a new member of the BH3 only pro-apoptosis family. This is supported by the observation that p193 expression promoted a prompt apoptotic response in NIH-3T3 cells. Immune cytologic analysis indicated that p193 is a cytoplasmic protein, and that co-expression of T-Ag resulted in the cytoplasmic localization of both proteins. p193-induced apoptosis occurs in G₁, and pulse chase experiments revealed that T-Ag is also localized in the cytoplasm (albeit transiently) at the same point of the cell cycle. The data are consistent with the conclusion that T-Ag possesses an anti-apoptosis activity, independent of p53 sequestration, which is actuated by T-Ag/p193 binding in the cytoplasm.

Accordingly, one aspect of the present invention concerns a method for modifying the cell cycle of a cell which involves modulating the level of p193 protein within the cell and/or interfering with the p193 protein signal transduction pathway in the cell. Particularly, increasing the wild-type pro-apototic, p193 activity can be used to induce apoptosis, and decreasing the level of pro-apoptotic p193 activity in the cell (including interfering with the p193 signal transduction pathway) can be used to suppress apoptosis and/or promote cellular proliferation. Increases in pro-apoptotic p193 activity can be achieved, for example, by expression of introduced DNA encoding a pro-apoptotic p193 protein. Decreases in pro-apoptotic p193 activity can be achieved, illustratively, by decreasing the level of expression of the native p193 of the cell (e.g. by antisense technology), and/or by interference with the pathway through which the native p193 acts, for example by the introduction of a dominant negative p193 protein which antagonizes at least a portion of the biological function of the native p193 protein. In certain aspects of the invention, methods for modifying the cell cycle of a cell include decreasing the level of expression of the native p193 protein of the cell and/or interfering with the p193 pathway, in conjunction with decreasing the level of expression of p53 protein in the cell or interfering with the p53 pathway, and/or in conjunction with increasing the level of expression of E1A protein in the cell.

In another aspect, the present invention provides an expression vector including nucleic acid encoding a p193 polypeptide. Such vectors can be used in inventive methods to genetically transduce host cells, and in the case of pro-apoptotic p193 polypeptides to induce apoptosis in the cells. In the case of p193 polypeptides with a dominant negative character, such transduction may be used to effectively suppress apoptosis or induce proliferation.

Another preferred embodiment of the invention provides an isolated p193 protein, preferably an isolated, recombinant p193 protein. Such proteins can be combined with an appropriate pharmaceutically acceptable carrier to produce pharmaceutical compositions, also constituting a part of the present invention. Such proteins can also be used in the preparation of inventive antibodies to p193.

The present invention also concerns a method for producing a p193 protein, comprising culturing a host cell having introduced DNA encoding a p193 protein under conditions suitable from expression of said introduced DNA.

The present invention provides a newly characterized apoptosis associated protein designated p193, and novel modified p193 proteins, including those exhibiting a dominant negative character; nucleotide sequences encoding such p193 proteins; products and processes involved in the cloning, preparation and expression of nucleotide sequences encoding p193 proteins; methods and materials for modifying the cell cycle in cells, for example regulating apoptosis and/or proliferation of cells; and methods for screening for pharmacological or other chemical agents for effect on cell cycle which involve assessing their impact on p193 or its signal transduction pathway in cells. Additional embodiments as well as features and advantages of the invention will be apparent from the descriptions herein.

### DESCRIPTION OF THE PREFERRED EMBODIMENTS

For the purpose of promoting an understanding of the principles of the invention, reference will now be made to certain preferred embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

As disclosed above, the present invention provides a novel apoptosis associated protein designated p193 and modified forms thereof; nucleotide sequences encoding p193 proteins; and products and processes involved in the cloning, preparation and expression of nucleotide sequences encoding p193 proteins.

SEQ. I.D. NO. 1 shows the nucleotide sequence and deduced amino acid sequence (see also SEQ. I.D. NO. 2) for mouse p193 as utilized in the Examples herein. SEQ. I.D. NO. 3 shows the nucleotide sequence and deduced amino acid sequence (see also SEQ. I.D. NO. 4) for human p193. In this regard, the term “nucleotide sequence,” as used herein, is intended to refer to a natural or synthetic sequential array of nucleotides and/or nucleosides, and derivatives thereof. The term amino acid sequence is intended to refer to a natural or synthetic sequential array of amino acids and/or derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

It will be understood that the present invention also encompasses the use of nucleotide sequences and amino acid sequences which differ from the specific p193 sequences disclosed herein, but which have substantial identity thereto and exhibit pro-apoptotic or proliferative activities as identified herein. Such sequences will be considered to provide p193 nucleic acid and p193 proteins for use in the various aspects of the present invention. For example, nucleic acid sequences encoding variant amino acid sequences are within the scope of the invention. Modifications to a sequence, such as deletions, insertions, or substitutions in the sequence, which produce “silent” changes that do not substantially affect the functional properties of the resulting polypeptide molecule are expressly contemplated by the present invention. For example, it is understood that alterations in a nucleotide sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also generally be expected to produce a biologically equivalent product.

It has also been discovered that modifications to the p193 sequence which substantially affect the functional properties of the resulting polypeptide can be made, and such changes are also expressly contemplated by the present invention. For example, modifications of the p193 amino acid sequence can be used to produce dominant-negative p193 proteins which antagonize at least a portion of the wild-type p193 activity, and which lead to suppression of apoptotic activity in the cells and/or an enhanced proliferative capacity of the cells.

In one manner of defining the invention, nucleic acid (e.g. DNA) may be used that has a coding sequence that differs from that set forth in SEQ. I.D. NO. 1 (nucleotides 62-5128) or SEQ. I.D. NO. 3 (nucleotides 87-5183), wherein the nucleic acid, or at least the coding portion thereof, will bind to nucleic acid having nucleotides 62-5128 of SEQ. I.D. NO. 1 or nucleotides 87-5183 of SEQ. I.D. NO. 3, or at least about nucleotides 62-3517 of SEQ. I.D. NO. 1 or about nucleotides 87-3615 of SEQ. I.D. NO. 3, under stringent conditions. Such nucleic acid will desirably encode a polypeptide having pro-apoptotic p193 activity, or a dominant-negative p193 polypeptide. “Stringent conditions” are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C.

In another manner of defining the invention, nucleic acid may be used that encodes a polypeptide that has an amino acid sequence which has at least about 70% identity, more preferably at least about 80% identity, most preferably a least about 90% identity, with the amino acid sequence set forth in SEQ. I.D. NO. 2 or in SEQ. I.D. NO. 4, or with at least one significant length (i.e. at least 40 amino acid residues) segment thereof, and which polypeptide possesses a pro-apoptotic p193 activity or a dominant-negative p193 character. The polypeptide may, for example, have an amino acid sequence which has at least about 70%, 80%, or 90% identity with at least about amino acid residues 1-1152 of SEQ. I.D. NO. 2 or about amino acid residues 1-1173 of SEQ. I.D. NO. 4, or with amino acid residues 1-1689 of SEQ ID. NO. 2 or amino acid residues 1-1698 of SEQ. I.D. NO. 4. Such polypeptides, especially when a functional pro-apoptotic protein is desired, will preferably include the characteristic p193 BH3 domain occurring at residues 1566 to 1572 of SEQ. I.D. NO. 2 or at residues 1575 to 1581 of SEQ. I.D. NO. 4:

Leu Lys Ala His Gly Asp Glu

Percent identity, as used herein, is intended to mean percent identity as determined by comparing sequence information using the advanced BLAST computer program, version 2.0.8, available from the National Institutes of Health, USA. The BLAST program is based on the alignment method of Karlin and Altschul, Proc. Natl. Acad. Sci. USA 87:2264-68 (1990) and as discussed in Altschul, et al., J. Mol. Biol. 215:403-10 (1990); Karlin and Altschul, Proc. Natl. Acad. Sci. USA 90:5873-7 (1993); and Altschul et al. (1997) Nucleic Acids Res. 25:3389-3402. Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Preferred default parameters for the BLAST program, blastp, include: (1) description of 500; (2) Expect value of 10; (3) Karlin-Altschul parameter λ=0.270; (4) Karlin-Altschul parameter K=0.0470; (5) gap penalties: Existence 11, Extension 1; (6) H value=4.94e⁻³²⁴; (6) scores for matched and mismatched amino acids found in the BLOSUM62 matrix as described in Henikoff, S. and Henikoff, J. G., Proc. Natl. Acad. Sci. USA 89:10915-10919 (1992); Pearson, W. R., Prot. Sci. 4:1145-1160 (1995); and Henikoff, S. and Henikoff, J. G., Proteins 17:49-61 (1993). The program also uses an SEG filter to mask-off segments of the query sequence as determined by the SEG program of Wootton and Federhen Computers and Chemistry 17:149-163, (1993).

In another form, nucleic acid may be used that includes a coding sequence that has at least about 70% identity with the coding portion of the nucleotide sequence set forth in SEQ. I.D. NO. 1 (nucleotides 62 to 5128) or in SEQ. I.D. NO. 3 (nucleotides 87 to 5183), or with at least one significant length (i.e. at least 100 nucleotides) segment thereof, and which nucleic acid encodes a polypeptide possessing pro-apoptotic p193 activity or dominant-negative 193 activity as identified herein. The nucleic acid may, for example, have a coding sequence which has at least about 70% at least about 80%, or at least about 90%, identity with nucleotides 62 to 5128 of SEQ. I.D. NO. 1 or with nucleotides 87 to 5183 of SEQ. ID. NO. 3, or at least with about nucleotides 62 to 3517 of SEQ. I.D. NO. 1 or about nucleotides 87 to 3615 of SEQ. I.D. NO. 3.

The p193 nucleotide sequence may be operably linked to a promoter sequence as known in the art to provide recombinant nucleic acid useful in a variety of applications including, for example, in the provision of vehicles such as vectors for functionally introducing the nucleic acid in to mammalian or other eukaryotic cells, such as cardiomyocytes, hepatocytes, smooth muscle cells, hemotpoietic stem cells, tumorogenic cells, and the like. As defined herein, a nucleotide sequence is “operably linked” to another nucleotide sequence (e.g. a regulatory element such as a promoter) when it is placed into a functional relationship with the other nucleotide sequence. For example, if a nucleotide sequence is operably linked to a promoter sequence, this generally means that the nucleotide sequence is contiguous with the promoter and the promoter exhibits the capacity to promote transcription of the gene. A wide variety of promoters are known in the art, including cell-specific promoters, inducible promoters and constitutive promoters. The promoters may be selected so that the desired product produced from the nucleotide sequence template is produced constitutively in the target cells. Alternatively, promoters, such as inducible promoters, may be selected that require activation by activating elements known in the art, so that production of the desired product may be regulated as desired. Still further, promoters may be chosen that promote transcription of the gene in one or more selected cell types, e.g. the so-called cell-specific promoters.

Expression vectors in accordance with the present invention can be designed to effectively increase wild-type p193 activity in a cell thus inducing apoptosis, or to interfere with wild-type p193 activity in a cell thus suppressing apoptosis and/or inducing proliferation. For example, expression vectors incorporating nucleic acid encoding a pro-apoptotic p193 polypeptide can be employed to increase apoptotic activity in a cell. On the other hand, vectors incorporating nucleic acid encoding a modified p193 polypeptide, for example truncation mutants of p193 exhibiting activity consistent with dominant negative (p193dn), can be used to interfere with wild-type p193 activity and thereby suppress apoptosis in the cell and/or induce proliferation of the cell. Genetic transduction of cells with vectors incorporating antisense (as) p193 nucleotide sequences can also be used to effectively suppress apoptotic activity and/or induce proliferation in the cells. Similarly, p193 antisense RNA may be administered to cells so as to decrease p193 and apoptotic activity and/or induce proliferation in the cells.

In a preferred aspect of the invention, the p193 nucleotide sequence is operably linked to a cell-specific promoter, for example, providing for constitutive expression of the nucleotide sequence in a selected cell type. Illustrative candidates for such promoters include cardiomyocyte-specific promoters such as the α-myosin heavy chain (α-MHC) promoter, the β-myosin heavy chain (β-MHC) promoter, the myosin light chain-2V (MLC-2V) promoter, the atrial natriuretic factor (ANF) promoter, and the like. Additional cell-specific promoters include liver-specific cells such as PePCK, albumin, transthyretin, and major urinary protein (MUP). Any cell type expressing endogenous gene, and its exressed ubiquitous, lung, heart, liver, eyes. Such constructs enable the expression of the p193 nucleic acid selectively in selected tissues.

Another aspect of the invention provides recombinant nucleic acid that includes a p193 nucleotide sequence encoding a p193 polypeptide operably linked to an inducible promoter. The p193 nucleotide sequence may, for instance, encode a pro-apoptosis polypeptide, such that expression and induces of apoptosis in cells, or an apoptosis-suppressing and/or proliferation-inducing polypeptide, such that expression suppresses apoptosis and/or promotes cellular proliferation. Using an inducible promoter, expression of the polypeptide encoded by the cells incorporating the nucleic acid can be upregulated in response to an inducing agent. Illustrative candidate inducible promoter systems include, for example, the metallothionein (MT) promoter system, wherein the MT promoter is induced by heavy metals such as copper sulfate; the tetracycline regulatable system, which is a binary system wherein expression is dependent upon the presence or absence of tetracycline; a glucocorticoid responsive promoter, which uses a synthetic sequence derived from the glucocorticoid response element and is inducible in vivo by administering dexamethasome (cells having the appropriate receptor); a muristerone-responsive promoter, which uses the ganodotropin-releasing hormone promoter and is inducible with muristerone (cells having the appropriate receptor); and TNF responsive promoters. Additional inducible promoters which may be used, and which are more preferred, include the ecdysone promoter system, which is inducible using an insect hormone (ecdysone) and provides complete ligand-dependent expression in mammals; the β-GAL system, which is a binary system utilizing an E. coli lac operon operator and the I gene product in trans, and a gratuitous inducer (IPTG) is used to regulate expression; and, the RU486 inducible system, which uses the CYP3A5 promoter and is inducible by RU486, a well defined pharmaceutical. These and other similar. inducible promoter systems are known, and their use in the present invention is within the purview of those skilled in the area.

One aspect of the present invention concerns the discovery that blocking p193 and p53 activity (by expression of dominant negative cDNA variants) protects against proliferation-induced apoptotic signals. This in turns renders cardiac myocytes responsive to the pro-proliferation signals, such signals encoded for example by E1A. Therapeutic approaches may be adopted which promote controlled regeneration of cardiac tissue, or alternatively controlled proliferation of engrafted cardiomyocytes, which rely upon the use of regulatable promoters to drive expression of the dominant negative cDNAs in addition to the growth promoting gene. An alternative approach may rely on pharmaceutical blockade of the p53 and/or the p193 pathways, in conjunction with expression of growth-promoting genes in combination with a regulatable promoter. For example, Gudkov and colleagues (Science (1999) 285; 1733-1737) developed an agent which inhibits p53-dependent transcriptional activation and apoptosis. Similar reagents to block p193 activity are readily generated by one skilled in the art. This approach has the advantage of an intrinsic cell cycle check-point in the event of illegitimate promoter activity (e.g. induction of promoter activity in the absence of inducing agent). Specifically, if the activity of one or both of the pro-apoptotic genes are blocked pharmaceutically, and that pharmaceutical(s) is withdrawn after regenerative growth is completed, illegitimate activation of the growth promoting gene would result in the apoptotic death of the cell because the anti-apoptotic activity would not be present. This approach may be used to provide a clinically safer modality to effect controlled cardiomyocyte proliferation in vivo.

An additional or alternative safeguard approach would encompass inclusion of a conditionally lethal gene in the expression cassette, as for example the well-known Herpes simplex virus thymidine kinase (HSV-TK) gene. The HSV-TK gene can incorporate normal nucleotides as well as the nucleotide analog gancyclovir at a high efficiency whereas mammalian thymidine kinase does not incorporate gancyclovir into cells at high efficiency. Incorporation of gancyclovir is cytotoxic. Thus, in this mode of operation, illegitimate activation of the regulatable promoter would result in expression of the anti-apoptosis (e.g. p53dn and p193dn) and pro-growth (e.g. E1A) genes, as well as the HSV-TK gene. Inappropriately growing cells (e.g. those where illegitimate promoter activity has occurred) can be eliminated by simple treatment with gancyclovir.

The present invention also concerns vectors which incorporate a p193 nucleotide sequence and which are useful in the genetic transduction of cells in vitro or in vivo. A variety of vector systems are suitable for these purposes. These include, for example, viral vectors such as adenovirus vectors as disclosed for example in Franz et al., Cardiovasc. Res. 35(3):560-566 (1997); Inesi et al., Am. J. Physiol. 274 (3 Pt. 1):C645-653 (1998); Kohout et al., Circ. Res. 78(6):971-977 (1996); Leor et al., J. Mol. Cell Cardiol. 28(10):2057-2067 (1996); March et al., Clin. Cardiol. 22(1 Suppl. 1):I23-29 (1999); and Rothman et al, Gene Ther. 3(10):919-926 (1996). Adeno-Associated Virus (AAV) vectors are also suitable, and are illustratively disclosed in Kaptlitt et al., Ann. Thora. Surg. 62(6):1669-1676 (1996); and Svensson et al., Circulation 99(2):201-205 (1999). Additional viral vectors which may be used include retroviral vectors (see e.g. Prentice et al., J. Mol. Cell Cardiol. 28(1):133-140 (1996); and Petropoulos et al., J. Virol. 66(6):3391-3397 (1992)), and Lenti (HIV-1) viral vectors as disclosed in Rebolledo et al., Circ. Res. 83(7):738-742 (1998). A preferred class of expression vectors will incorporate the p193 nucleic acid operably linked to a cardiomyocyte-specific promoter, such as one of those identified above. Still further, AAV vectors are highly compatible for use in transfection of myocardial and other cells and tissue, and are preferred from among those identified above.

In accordance with the invention, cells can also be genetically transduced with p193 nucleic acid in vitro or in vivo using liposome-based transduction systems. A variety of liposomal transduction systems are known, and have been reported to successfully deliver recombinant expression vectors to a variety of cells. Illustrative teachings may be found for example in R. W. Zajdel, et al., Developmental Dynamics. 213(4):412-20 (1998); Y. Sawa, et al., Gene Therapy.5(11):1472-80 (1998); Y. Kawahira, et al., Circulation 98(19 Suppl):II262-7; discussion II267-8 (1998); G. Yamada, et al., Cellular & Molecular Biology 43(8):1165-9 (1997); M. Aoki, et al., Journal of Molecular & Cellular Cardiology 29(3):949-59 (1997); Y. Sawa, et al., Journal of Thoracic & Cardiovascular Surgery 113(3):512-8; discussion 518-9 (1997); and I. Aleksic, et al., Thoracic & Cardiovascular Surgeon 44(2):81-5 (1996). Thus, liposomal recombinant expression vectors including p193 DNA can also be utilized to tranduce cells in vitro and in vivo for the purposes described herein.

Nucleic acid constructs can be used for example to introduce nucleotide sequences encoding a p193 protein into cells in vivo or in vitro, to achieve a level of intracellular p193 activity that is increased relative to the native level of the cells. Such increased activity can induce apoptosis in the cells. Induction of apoptotic activity can be evidenced, for example, by cell death and other characteristic morphological changes such as cell shrinkage and nuclear condensation and fragmentation. Alternatively or in addition, purified (e.g. purified recombinant) p193 protein may be introduced into cells to increase p193 activity (e.g. by fusogenic liposomes or other macromolecular delivery systems), or the cells can be treated with pharmacologic agents which increase p193 activity, to provide increased apoptotic activity to the cells.

Nucleic acid constructs can also be used to introduce modified p193 nucleotide sequences into cells in vivo or in vitro, wherein the sequences provide characteristics of a dominant negative gene and effectively antagonize wild-type p193 activity, resulting in as for example a suppression of apoptosis and/or an increase in the proliferative capacity of the cells. In a similar approach, a dominant negative p193 protein or another molecule can be introduced into the cells which interferes with or antagonizes wild-type p193 activity, and thereby suppresses apoptosis and/or induces proliferation in the cells. Ilustratively, vectors incorporating antisense (as) p193 nucleotide sequences can be used, and/or small synthetic organic molecules serving as pharmacologic agents can be used, to effectively interfere with the expression of or the activity of wild-type p193 protein.

The present invention makes available methods which can be applied in vitro or in vivo for research, therapeutic, screening or other purposes. Methods for the in vitro culture of cells expressing introduced p193 DNA (in sense or antisense orientation) can be used, for example, in the study and understanding of the cell cycle, in screening for chemical or physical agents which modulate p193 activity or other aspects of the cell cycle, or in the culture of cells having suppressed apoptotic activity and/or increased proliferative potential for subsequent engraftment into mammals, including humans.

Cells to be cultured in accordance with the invention can be derived from a variety of sources. For example, they may be harvested from a mammal for culture and subsequent engraftment into that mammal (autografts) or another mammal of the same species (allografts) or a different species (xenografts). Cardiomyocyte or other cells may also be derived from the differentiation of stem cells such as embryonic stem cells, somatic stem cells or other similar pluripotent cells. General methodology for such derivations is disclosed in U.S. Pat. Nos. 5,602,301 and 5,733,727 to Field et al. In this regard, when so derived, the genetic modification to incorporate the p193 nucleic acid may take place at the stem cell level, for instance utilizing one or more vectors to introduce the p193 nucleic acid operably linked to a tissue-specific promoter, and nucleic acid enabling the selection of a target cell type from other cells differentiating from the stem cell and/or at a differentiated level e.g., including a selectable marker gene operably linked to a tissue-specific promoter. Nucleic acid enabling selection of transduced from non-transduced stem cells may also be used in such strategies. Such selection of the stem and/or differentiated cell types may be achieved, illustratively, utilizing a gene conferring resistance to an antibiotic (e.g. neomycin or hygromycin) or other chemical agent operably linked to an appropriate promoter.

Using stem-cell derived cells, the genetic modification to incorporate the p193 and potentially other nucleic acid may also occur after differentiation of the stem cells. For example, a differentiated cell population enriched in cardiomyocytes or another target cell type, for instance containing 90% or more of the target cell type, may be transformed with a vector having p193 nucleic acid (especially antisense or including a dominant negative mutation) operably linked to a promoter (optionally tissue specific), as described above. The same or a different vector may also be used to introduce other functional nucleic acid to the cells, for example providing a reporter gene and/or selectable marker, or providing for the expression of a growth factor and/or another cell cycle regulatory protein.

Illustratively, in certain embodiments of the invention, decreasing the level of p193 protein or interfering with the p193 signal transduction pathway can be used in conjunction with other means of effecting the cell cycle. For example, such modifications of p193 and/or its pathway (effected e.g. by an introduced antisense p193 nucleic acid or a nucleic acid having a dominant negative mutation) can be used in combination with a p53 nucleic acid (especially antisense or a dominant negative mutation), an E1A nucleic acid, or a combination of the two. Still further, such modifications of p193 and/or its pathway may be used in conjunction with other methods of relaxing or facilitating the G₁/S transit, for example by manipulating key regulators at the restriction point of the cell cycle such as inhibiting RB family members, overexpressing D-type cyclin or cyclin-dependent kinase activities, inhibiting cyclin-dependent kinase inhibitors, overexpressing downstream targets, and the like.

In one mode of carrying out the invention, left ventricular, right ventricular, left atrial, or right atrial cardiomyocytes, or a mixture of some or all of these, may be genetically modified in vitro to incorporate anti-apoptotic and/or proliferative p193 nucleic acid using a suitable vector as disclosed above. Cells to be genetically transduced in such protocols may be obtained for instance from animals at different developmental stages, for example fetal, neonatal and adult stages. Suitable animal sources include mammals such as bovine, porcine, equine, ovine and murine animals. Human cells may be obtained from human donors or from a patient to be treated. The modified cardiomyocytes may thereafter be implanted into a mammal, for example into the left or right atrium or left or right ventricle, to establish a cellular graft in the mammal. Implantation of the cells may be achieved by any suitable means, including for instance by injection or catheterization. In addition to the p193 nucleic acid, the cells may also be modified in vitro to contain other functional nucleic acid sequences which can be expressed to provide other proteins, for example or one or more additional cell cycle regulatory proteins. In one preferred embodiment, cells are modified with nucleic acid encoding p193 and with nucleic acid encoding at least one other cell cycle regulatory protein, for example combining forced expression of p193 and p53 dominant (Mowat, M., Nature Vol. 314, p. 633-636 (1985); Munroe, D. G. Mol. Cell. Biol., Vol. 10, 3307-3313 (1990) so as to suppress apoptosis in the cells.

Cells for culture, and potential implantation, may also be obtained from a transgenic animal (especially mammal) expressing introduced p193 nucleic acid. Using known techniques, transgenic animals which harbor introduced p193 nucleic acid in essentially all of their cells can be raised, and used as sources for harvesting culturable cells (e.g. cardiomyocytes), tissues or organs, or may be used as animal models for research or screening purposes. For instance, transgenic bovine, porcine, equine, ovine or murine animals may be used as sources for cells, tissues or organs, or as animal models for study. Illustratively, transgenic animals having reduced levels of wild-type p193 protein and/or expressing an introduced dominant negative p193 protein, can be used as a source for apoptotically-suppressed and/or proliferatively enhanced cells, tissue or organs, which will be protected against fibrosis or other similar damage. Such materials will thus possess significant advantages for use in transplantation into other animals, such as humans.

The present invention also provides for the genetic modification of cells in vivo to increase p193 activity (using pro-apoptotic protein) or decrease p193 activity (using p193dn) in the cells (impacting transduction pathway). An expression vector containing the p193 nucleic acid, for instance one as described above, may be delivered to tissue of a recipient mammal, to achieve transduction of cells in the tissue. In preferred modes, the p193 nucleic acid in such vectors will be operably linked to a tissue-specific promoter, for instance a cardiomyocyte-specific promoter. The delivery of the vector can be suitably achieved, for instance, by injection, catheterization, or infusion into the blood stream, or by other known means. It will be understood that any mode of delivery which enables the establishment of transduced cells within the recipient mammal is contemplated as being within the present invention. A single delivery of the vector may be used, or multiple deliveries nearly simultaneous or over time may be used, in order to establish a substantial population of transduced cells within the recipient. The transduced cells will then express the encoded p193 polypeptide, for instance under the control of a constitutive, inducible or tissue-specific promoter, and thereby exhibit a suppressed or induced level of apoptosis.

The implantation of cells cultured in vitro or the delivery of the vector for in vivo genetic transduction may be directed to a selected site or sites within the recipient. For example, in the case of apoptosis-suppressed and/or proliferatively enhanced cardiomyocyte engraftment or corresponding in vivo transduction, such site or sites may be in the left or right atrium or left or right ventricle of the recipient, or any combination of these. Commonly, the implantation or delivery site or sites will occur in the left or right ventricle of the recipient. The site(s) may, for instance, be one(s) in which there is a need for additional viable cells, for example in a damaged or diseased area of the heart such as in cases of myocardial infarcts and cardiomyopathies. The site(s) may also be targets for the delivery of other proteins such as growth factors, e.g. nerve growth or angiogenic factors, via expression in the grafted or in vivo transduced cells.

Cellular engraftment and/or in vivo genetic modification in accordance with the invention can be used, for example, to deliver therapy to mammals, including humans. A variety of ex vivo cellular transplantation and implantation techniques and gene therapy techniques are thus contemplated as forming a part of the invention. For example, these techniques may be used to provide cells in the mammal having a reduced level of wild-type p193 protein and/or having a disrupted or partially disrupted p193 signal transduction pathway, the cells thereby exhibiting decreased apoptotic activity and/or an enhanced proliferative capacity. Illustratively, such an approach may be used to target an improvement or protection of the contractile function of the heart of the patient, for example in the treatment of contractile losses due to infarcts or cardiomyopathies. They may also be used to target an improvement and/or protection of the function of other tissue or organs in the patient, for example the liver or lungs of the patient. The use of a p193 protein having a dominant negative mutation will be especially advantageous for such purposes. In addition, the delivery of pro-apoptotic p193 protein to cells, for example by in vivo genetic transduction with an appropriate p193 nucleic acid and consequent expression of the pro-apoptotic protein, can be used to promote apoptosis in cells in which apoptosis is desired, for example in the case of inappropriately proliferative cells.

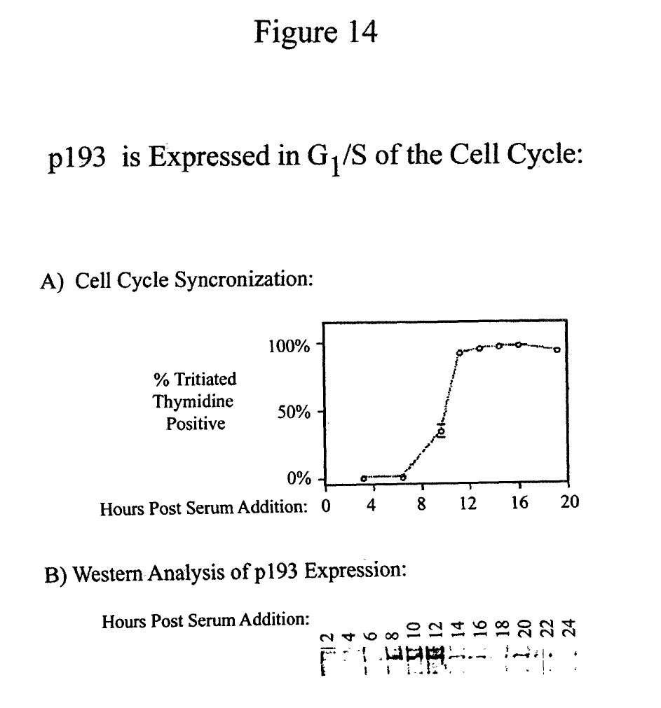

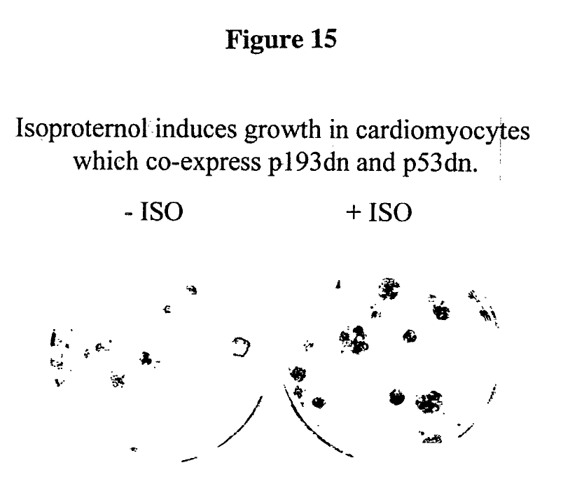

The present invention also provides access to antibodies having specificity to one or more epitopes present on the p193 peptide, or an idiotype on the p193 (see e.g. FIG. 14 and accompanying discussion in Examples). Such antibodies can be polyclonal or monoclonal, and can be made with the p193 polypeptide or fragment thereof as the immunogen. In this regard, the term “antibody” (Ab) or “monoclonal antibody” (Mab) as used herein is meant to include intact molecules as well as fragments thereof capable of binding an antigen. Antibodies to p193 can be used, for example, to detect the presence of the p193 protein in a human or other mammalian tissue sample. This may involve contacting the sample with a detectably labeled antibody and detecting the label, thereby establishing the presence of the p193 protein in the sample. Detection can be carried out by imaging in vivo. The p193 protein can also be detected by known immunoassay techniques, including, for example, RIA, ELISA, etc., using appropriate antibodies according to the invention.

Antibodies of the invention can be prepared by any of a variety of known methods. For example, cells expressing the p193 protein can be administered to an animal in order to induce the production of serum containing polyclonal antibodies that are capable of binding the p193 protein. For example, the p193 protein or fragment thereof is chemically synthesized and purified by HPLC to render it substantially free of contaminants. Such a preparation is then introduced into an animal in order to produce polyclonal antisera of high specific activity.

Polyclonal antibodies can be generated in any suitable animal including, for example, mice, rabbits or goats. The p193 immunogenic peptide or fragment thereof can be injected by itself or linked to an appropriate immunoactivating carrier.

Monoclonal antibodies can be prepared in various ways using techniques well understood by those having ordinary skill in the art. For example, monoclonal antibodies can be prepared using hybridoma technology (Kohler, et al., Nature 256:495 (1975); Kohler, et al., Eur. J. imninol. 6:511 (1976); Kohler, et al., Eur. J. Immunol. 6:292 (1976); Hanmerling, et al., In: Monoclonal Antibodies and T-Cell Hybridomas, Elsevier, N.Y., pp. 563-681 (1981)); Roger H. Kennett, et al., Eds., Monoclonal Antibodies—Hybridomas: A New Dimension in Biological Analysis, Plenum Press (1980). In general, such procedures involve immunizing an animal with the present p193 protein, or a fragment thereof. Splenocytes from such animals are extracted and fused with a suitable mycloma cell line. Any suitable mycloma cell line may be employed in accordance with the present invention. After fusion, the resulting hybridoma cells are selectively maintained in HAT medium, and then cloned by limiting dilution as described by Wands, et al., Gastroenterol. 80:225-232 (1981). The hybridoma cells obtained through such a selection are then assayed to identify clones which secrete antibodies capable of binding the p193 protein.

These and other general techniques in relation to the production and use of antibodies will be apparent to and readily utilizable by those of ordinary skill in the art to produce and use p193 antibodies of the present invention.

For the purpose of promoting a further understanding of the present invention and its advantages, the following specific Examples are provided. It will be understood that these Examples are illustrative, and not limiting, of the invention.

### EXAMPLES

### Examples 1-3: Methods

### Example 1

### Isolation and Sequence Analysis of p193 Proteins

AT-2 cardiomyocytes were homogenized in 20 ml of NET, pre-cleared with protein A sepharose beads, and mixed with anti-T-Ag monoclonal antibody PAb419 (90 min., 4° C.). Immune complexes were collected with Protein A-sepharose, displayed on polyacrylamide gels and visualized by staining with Coomassie Brilliant Blue. The region of the gel containing p193 was excised, alkylated with isopropylacetamide, and digested with F-trypsin (0.2 μg trypsin, 37° C., 17 hrs) as described (Shevchenko, A., Wilm, M., Vorm, O., and Mann, M. (1996) Anal. Chem. 68, 850-858). The peptides were then extracted with 5% formic acid/50% acetonitrile and separated on a C18 0.32×100 mm capillary column (LC Packing, Inc.). An aliquot of each of the isolated HPLC fractions was applied to a pre-made spot of matrix (0.5 ml of 20 mg/ml α-cyano-4-hydroxycinammic acid +5 mg/ml nitrocellulose in 50% acetone/50% 2-propanol) on the target plate. Ions were formed by matrix-assisted laser desorption/ionization with a nitrogen laser, 337 nm. Spectra were acquired with a PerSeptive Biosystems Voyager Elite time-of-flight mass spectrometer, operated in linear delayed extraction mode. Subsequently, fragment ions for selected precursor masses were obtained from post-source decay (PSD) experiments (Kaufmnan, R., Kirsch, D. and Spengler, B. (1994) International J. Mass Spec. and Ion Proc. 131, 355-385). Automated protein sequencing was performed on a model 470A Applied Biosystems sequencer equipped with an on-line PTH analyzer using modified cycles as described (Henzel, W. J., Grimley, C., Bourell, J. H., Billeci, T. M., Wong, S. C. and Stults, J. T. (1994) Methods: A companion to Methods in Enzymology 6, 239-247). Peaks were integrated with Justice Innovation software using Nelson Analytic 760 interfaces. Sequence interpretation was performed on a DEC 5900 (Henzel, W. J., Rodriguez, H., and Watanabe, C. (1987) J. Chromatogr. 404, 41-52).

### Example 2

### Isolation and Molecular Analysis of p193 cDNAs

p193 cDNAs were isolated from an adult heart cDNA library generated from C3HeB/FeJ inbred mice (Kim, K. K., Daud, A. I., Wong, S. C., Pajak, L., Tsai, S. C., Wang, H., Henzel, W. J., and Field, L. J. (1996) J. Biol. Chem. 271, 29255-29264). Plaque hybridizations, phage DNA isolation and subcloning were performed using standard methodologies (Sambrook, J., Fritsch, E. F., and Maniatis, T. (1989) Molecular cloning, Cold Spring Harbor Laboratory Press, Cold Spring Harbor, N.Y.). Sequence was determined for both strands of the cDNA clone examined using the dideoxy chain terminating approach (Sequenase, United States Biochemicals, Cleveland, Ohio).

To demonstrate p193/T-Ag binding, a full length p193 cDNA was subcloned into the pcDNA3.1/Myc-His expression vector (Invitrogen, Carlsbad Calif.) such that the epitope tag was incorporated into the C-terminus of the molecule (construct designated CMV-p193myc). A T-Ag cDNA was subcloned into pcDNA3.1 expression vector (which lacks the epitope tag; construct designated CMV-T-Ag). For IP/Western analyses, NIH-3T3 cells (ATCC, Rockville Md.) were co-transfected with CVM-5-Ag and CMV-p193myc using the calcium phosphate approach (Sambrook, J., Fritsch, E. F., and Maniatis, T. (1989) Molecular cloning, Cold Spring Harbor Laboratory Press, Cold Spring Harbor, N.Y.). Protein (1 mg) prepared from the transfected cells was reacted with an anti-T-Ag (PAb419), an anti-myc (9E10, Santa Cruz Biotech.), or an IgG subtype-matched non-specific antibody (anti-GST, Pharmacia), and the resulting immune complexes were subjected to Western blot analysis. 100 μg of total protein from non-transfected and transfected cells were included as controls. The blots were probed with an anti-myc (9E10) or anti-T-Ag (PAb416) antibody, and signal was developed using the ECL method. To demonstrate p193/T-Ag binding in vitro, ³⁵S-methionine labeled in vitro transcription/translation (TNT kit, Promega) product obtained from a full length p193 cDNA subcloned into pBluescript IISK (Stratagene, LaJolla Calif.) was mixed with 1.2 μg of recombinant SV40 T-Ag (Molecular Biology Resource), and reacted with anti-T-Ag (PAb419) or an IgG subtype-matched nonspecific control antibody (anti-MAP kinase #D2, Santa Cruz Biotech.). Immune complex was then visualized via autoradiography (p193) or Western blotting (T-Ag) as described above.

For Northern blots, 10 μg of total RNA was denatured with glyoxal, displayed on agarose gels, transferred to Genescreen (NEN) and reacted with a nick-translated full-length p193 cDNA as described (Sambrook, J., Fritsch, E. F., and Maniatis, T. (1989) Molecular cloning, Cold Spring Harbor Laboratory Press, Cold Spring Harbor, N.Y.). For mapping the p193 binding site on T-Ag, in vitro translation products from a full length p193 cDNA clone and various t-Ag deletion constructs were mixed, and immune complex was generated with an N-terminal specific anti-T-Ag monoclonal antibody (PAb419). Immune complex was then visualized by autoradiography. The various T-Ag deletion constructs were generated via PCR amplification using oligonucleotide primers incorporating stop codons or base pair substitutions as indicated in the text. The fidelity of each construct was confirmed by sequence analysis.

### Example 3

### Functional Analyses of p193

For FACS analyses, NIH-3T3 cells transfected with either CMV-β-GALmyc or CMV-p193myc were labeled with Hoechst, trypsinized and fixed in 5% acetic acid in ethanol, rehydrated in PBS and reacted with FITC-conjugated anti-myc antibody (9E10, Oncogene Sciences) as described (Esser, C., Gottlinger, C., Kremer, J., Hundeiker, C., and Radbruch, A. (1995) Cytometry 21, 382-386; Brown, D. R., Thomas, C. A., and Deb, S. P. (1998) EMBO J., 17, 2513-2525). The DNA content of FITC-positive cells was then analyzed on a Becton Dickinson FACS-PLUS instrument. Immune cytologic analyses were as described (44), and images were captured using a BioRad laser scanning confocal microscope or photographed directly using conventional light or fluorescent microscopy.

For the cell death time course experiment, NIH-3T3 cells synchronized by two rounds of serum depletion (starvation media contained 0.1% FBS in DMEM) were transfected with either CMV-p193myc or CMV-βGALmyc using Lipofectin (Gibco-Life Sciences, Grand Island N.Y.) for 24 hrs. The cultures were then rinsed with PBS, and cultured for an additional 6 hrs in starvation media. Media containing ³H-thymidine (26 Ci/mmol, Amersham, Buckinghamshire, England) and 10% FBS in DMEM was then added, and cells were processed for autoradiography and immune cytology at various points thereafter as described (Klug, M. G., Soonpaa, M. H., Koh, G. Y., and Field, L. J. (1996) J. Clin. Invest. 98, 216-224). To localize T-Ag during the cell cycle, subconfluent culture of AT-2 cells in DMEM containing 10% FBS received a 40 minute pulse of ³H-thymidine. The cells were then rinsed, and then cultured with DMEM containing 10% FBS. The cells were then processed for autoradiography and immune cytology at various points thereafter as described (Klug, M. G., Soonpaa, M. H., Koh, G. Y., and Field, L. J. (1996) J. Clin. Invest. 98, 216-224). For the colony growth assay, NIH-3T3 cells transfected with CMV-null, CMV-p193s or CMV-p193as were selected in G418 for 15 days (the expression vectors also encoded a CMV-neo cassette). The dishes were then fixed and stained with gentian violet.

### Examples 1-3: Results

Cloning of p193

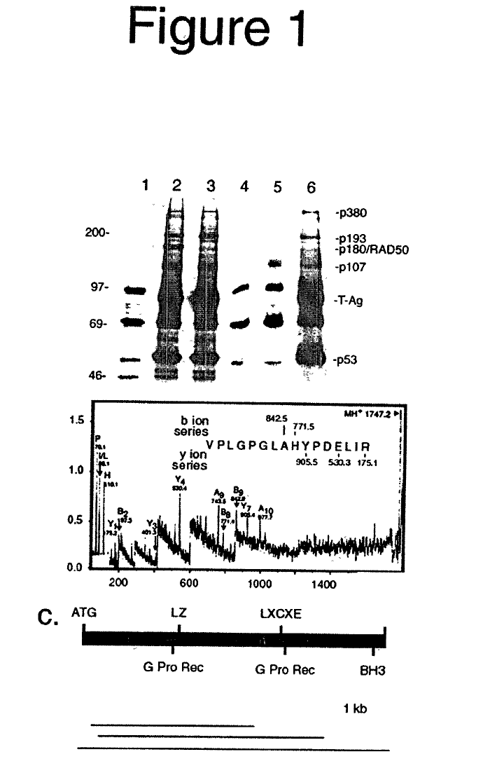

To identify the T-Ag binding proteins in cardiomyocytes, immune complexes were generated using protein prepared form ³⁵S-methionine labeled AT-2 cells, a cell line derived form the transgenic heart tumors (Daud, A. I., Lanson, N. A., Jr., Claycomb, W. C., and Field, L. J. (1993) Am. J. Physiol 264, H1693-700). Proteins with apparent molecular weights of 380, 193 and 120 kd (see FIG. 1a) were detected in immune complex generated with either anti-T-Ag (PAb419, lane 3) or anti-p53 (PAb421 and PAb246, lanes 2 and 6, respectively) monoclonal antibodies. These proteins were not present in immune complexes generated with IgG subtype-matched nonspecific control antibodies (DYS 1, lane 1; PAb240, lane 5), nor in controls lacking primary antibody (lane 4). Previous studies have shown that the 120 kd protein is p107 (45, 46), and that the 180 kd protein (present only in PAb421 anti-p53 immune complex) is the murine homologue of RAD50, a protein involved in the repair of dsDNA breaks in yeast (Kim, K. K., Daud, A. I., Wong, S. C., Pajak, L., Tsai, S. C., Wang, H., Henzel, W. J., and Field, L. J. (1996) J. Biol. Chem. 271, 29255-29264). The 380 kd protein has not yet been characterized.

To clone p193, large scale anti-T-Ag immune complex preparations were resolved on polyacrylamide gels and visualized by Coomassie blue staining. The region containing p193 was excised, digested with trypsin in situ, fractionated by HPLC and analyzed by mass spectroscopy using post source decay (PSD, FIG. 1b). Information obtained from the PSD experiment was used to search a protein sequence database using a modified version of Frag-Fit. The search indicated that p193 was homologous to a previously identified open reading frame of unknown function isolated from a human immature myeloid cell line (Nomura, N., Nagase, T., Miyajima, N., Sazuka, T., Tanaka, A., Sato, S., Seki, N., Kawarabayasi, Y., Ishikawa, K., and Tabata, S. (1994) DNA Res. 1, 251-262). Reverse transcriptase-polymerase chain reaction was used to generate a short cDNA clone spanning the region homologous to the largest p193 peptide. This clone was then used to screen an adult mouse heart cDNA library.

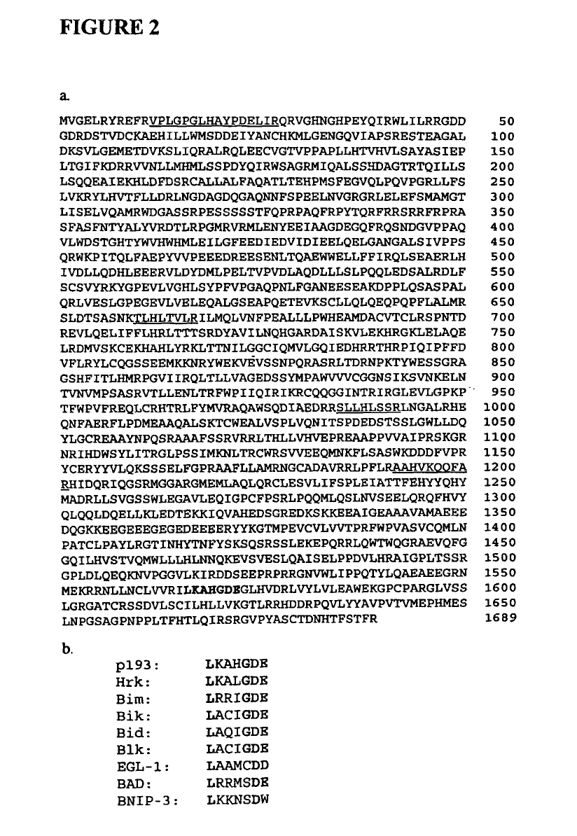

Six overlapping cDNA clones were ultimately obtained (FIG. 1c). Sequence analysis revealed an open reading frame 5067 nucleotides in length which encoded a protein of 1689 amino acid residues and with a deduced molecular weight of 192,346 d (FIG. 2a). All of the p193 proteolytic peptides identified in the PSD experiment were present in the deduced amino acid sequence of the cDNA clones. Analysis of the sequence revealed the presence of a leucine zipper at amino acid residues 593-619, an LXCXE motif (the consensus sequence for binding to retinoblastoma family proteins, see Gibson, T. J., Thompson, J. D., Blocker, A., and Kouzarides, T. (1994) Nucleic. Acids. Res. 22, 946-952) at amino acid residues 1052-1056, and two G protein receptor motifs at amino acid residues 1566-1572: No other BH domains were present in p193. Sequence alignment of the BH3 domain of p193 and those of the BH3 only family members is shown in FIG. 2b.

Cloned p193 Binds to T-Ag

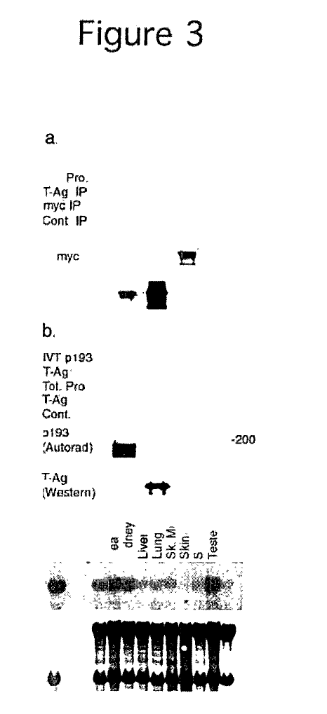

To confirm that these clones encoded the 193 kd T-Ag binding protein, a full length p193 cDNA was subcloned into a CMV-promoted expression vector which incorporated a short myc-epitope tag at the C-terminus. The resulting clone, designated CMV-p193myc, was co-transfected alone with CMV-T-Ag (an expression construct encoding T-Ag) into NIH-3T3 cells. Protein prepared from cells 24 hrs. post transfection was subjected to Immune Precipitation/Western blot analyses using anti-T-Ag and anti-myc antibodies (FIG. 3a). p193myc was detected in anti-T-Ag immune complex, and T-Ag was detected in anti-myc immune complex. Neither protein was present in immune complex generated with an AgG subtype-matched nonspecific control antibody. Immune Precipitation analyses of mixtures of in vitro translated p193 and recombinant T-Ag were also performed (FIG. 3b). Radiolabeled p193 was present in immune complex generated with anti-T-Ag antibody, but not in immune complex generated with an IgG subtype-matched nonspecific control antibody, confirming that the 193 kd T-Ag binding protein was successfully cloned. Northern blots revealed a somewhat restricted pattern of p193 expression in adult mouse tissues (FIG. 3c). Relatively high levels of p193 mRNA were detected in the heart, as might be anticipated given that the protein was originally identified in cell lines derived from cardiac tumors.

193 Binds to the N-terminus of T-Ag

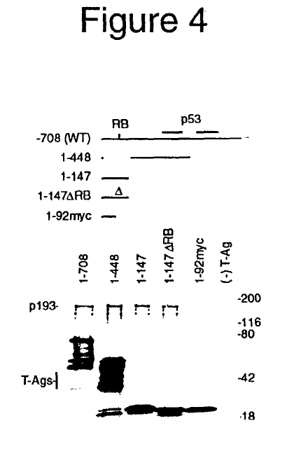

To identify the region of T-Ag which binds to p193, in vitro translation products from a series of T-Ag deletion constructs were mixed with full length in vitro translated p193, and immune complexes generated with anti-T-Ag antibody were resolved on polyacrylamide gels and visualized by autoradiography (FIG. 4). p193 was present in immune complex generated with T-Ag mutants with deletions encompassing as much as amino acid residues 147 through 708, indicating that the p193 binding site resides within T-Ag amino acid residues 1 through 147. In contrast, p193 was not present in immune complex generated with a T-Ag mutant in which amino acid residues 92 through 708 where deleted, indicating that the C-terminal boundary of the binding site lies within T-Ag amino acid residues 107 and 108 which disrupt binding of RB family members did not effect p193 binding (FIG. 4, construct 1-147▴ RB). Thus, p193 binds to the N-terminal region of T-Ag distinct from the RB family member binding site.

Expression of p193 Promotes Apoptosis

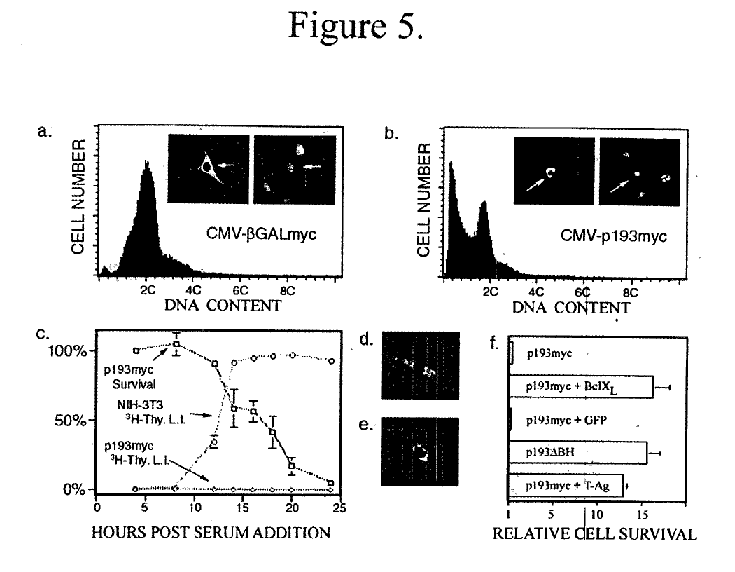

To determine the effects of p193expression on cell growth, NIH-3T3 cells were transfected with either CMV-βGALmyc (an expression construct encoding β-galactosidase with a myc-epitope tag) or CMV-p193myc. At 48 hrs. post-transfection, FACS analyses using a FITC-conjugated anti-myc antibody revealed that most cells expressing CMV-βGALmyc had a normal 2C DNA content (FIG. 5a; the inset shows a CMV-βGALmyc transfected cell: image on the left shows anti-myc immune fluorescence (see arrow), image on right shows nuclear morphology via Hoechst fluorescence (see arrow)). In contrast the preponderance of cells expressing CMV-p193myc exhibited hypodiploid DNA content, indicative of apoptotic cell death (FIG. 5b). Visual inspection of the cultures confirmed that the bulk of the transfected cells were dying and had markedly condensed chromatin (FIG. 5B inset: image on the left shows anti-myc immune fluorescence (see arrow), image on right shows nuclear morphology via Hoechst fluorescence (see arrow)). Thus expression of p193 can induce apoptosis.

To determine at what point in the cell cycle p193 induced cell death, serum-starved NIH-3T3 cells were transfected with CMV-p193myc. Media containing serum and ³H-thymidine was then added, and the cultures were processed for anti-myc immune cytology and autoradiography at various time points thereafter. Most cells expressing CMV-p193myc were dead by 20 hrs. post-serum replenishment (FIG. 5c, trace with square symbols), and DNA synthesis never reinitiated in these cells (FIG. 5c, trace with diamond symbols. This suggests that p193-induced apoptosis occurs during G₁. In contrast, the preponderance of non-transfected cells on the same chamber slide reinitiated DNA synthesis by 14 hrs. post-serum replenishment (FIG. 5c trace with circle symbols), thus establishing the fidelity of the synchronization protocol. In control experiments cells expressing CMV-βGALmyc reinitiated DNA synthesis at a rate comparable to the non-transfected cells (not shown), indicating that transgene expression per se did not impact on cell cycle progression or viability. p193myc immune reactivity in the synchronized cultures was initially localized uniformly throughout the cytoplasm, but became restricted to the perinuclear region prior to the onset of cell death (FIGS. 5d and e, respectively). Bax, a well characterized pro-apoptosis protein, undergoes a similar cytoplasmic to perinuclear redistribution during apoptosis (Hsu, Y. T., Wolter, K. G., and Youle, R. J. (1997) Proc. Natl. Acad. Sci. USA 94, 3668-3672). Finally, it is of interest to note that p193myc expressing cells are viable if cell cycle progression is blocked; >90% of p193 expressing cells were viable at 40 hrs. post transfection if maintained under low serum conditions. This suggests that some degree of cell cycle progression is needed to actuate apoptosis.

Cell death induction by BH3 only family members can be antagonized by co-expression of pro-survival members of the Bcl-2 family. To determine if p193 shares this trait, NIH-3T3 cells were transfected with CMV-p193myc alone or co-transfected with CMV-p193myc and CMV-Blc-X_{L} (a construct encoding human Bcl-X_{L}). The preponderance of cells transfected with p193 alone were dead at 68 hrs. post transfection, whereas co-transfection with Bcl-X_{L} markedly antagonized p193-induced apoptosis (FIG. 5f). In control experiments, virtually no viable cells were seen following co-transfection with CMV-p193myc and CMV-GFP (an expression construct encoding green fluorescent protein), indicating that co-expression of two CMV-driven constructs does not abate p193-induced cell death (FIG. 5f).

To establish the importance of the BH3 domain in p193-induced apoptosis, the CMV-p193myc expression construct was modified such that amino acid residues 1563 through 1576 were deleted (VRILKAHGDEGLHV). This modification resulted in the deletion of the BH3 motif (amino acid residues 1566-1572,), and the resulting construct was designated CMV-p193[delta symbol]BH. NIH-3T3 cells transfected with CMV-p193[delta symbol]BH were viable (FIG. 5f). Indeed, the survival was similar to that obtained for cells co-transfected with CMV-p193 plus CMV-Bcl-XL. Thus the BH3 domain is required for p193-mediated apoptosis.

T-Ag is Transiently Localized in the Cytoplasm During M and G₁

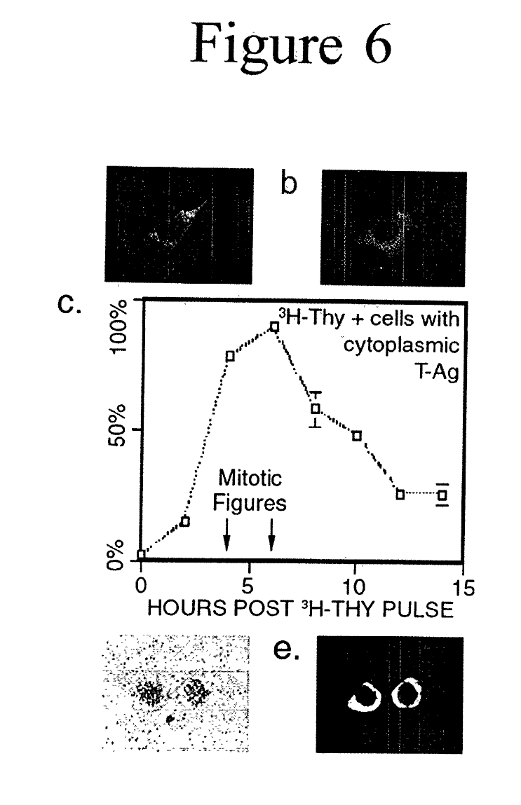

Given that p193 was originally identified as a T-Ag binding protein, and that T-Ag is a nuclear oncoprotein, the cytoplasmic/perinuclear localization of p193myc was somewhat surprising. To further address this paradox, NIH-3T3 cells co-transfected with CMV-193myc and CMV-T-Ag were examined. Survival was greatly enhanced in cells co-expressing p193 and T-Ag (FIG. 5f), indicating that, like Bcl-X_{L}, T-Ag can antagonize p193-induced apoptosis. Moreover immune cytologic analyses indicated that p193myc and T-Ag co-localized to the cytoplasm in the majority (ca. 63%) of the co-transfected cells (FIGS. 6a and brespectively). In contrast, co-transfection with CMV-βGALmyc and CMV-T-Ag did not result in prominent cytoplasmic T-Ag immune reactivity (not shown). These results raised the possibility that p193-T-Ag binding might normally occur in the cytoplasm.

Cytoplasmic T-Ag immune reactivity has previously been noted in mitotic cells (Stenman, S., Zeuthen, J., and Ringertz, N. R. (1975) International Journal. Of. Cancer 15, 547-554; Davis, D. and Wynford-Thomas, D. (1986) Experimental. Cell Research. 166, 94-102). A pulse chase experiment was used to determine how long T-Ag persists in the cytoplasm of AT-2 cardiomyocytes. Sub-confluent cultures received a 40 min. pulse of ³H-thymidine (to mark cells in S-phase) followed by a chase with radioisotope free media. The cultures were processed for anti-T-Ag immune cytology and autoradiography at various time points thereafter. Significant percentages of the thymidine positive cells exhibited cytoplasmic T-Ag immune reactivity from 4 hrs. through 10 hrs. post S-phase (FIG. 6c). In contrast, ³H-thymidine positive cells with mitotic figures were only observed at 4 and 6 hrs. post S-phase. Thus, cytoplasmic T-Ag localization persisted through cytokinesis and well into G₁. This point is further illustrated by the presence of ³H-thymidine positive daughter cells with cytoplasmic T-Ag immune reactivity at 8-12 hrs. post S-phase (FIGS. 6d and e). Thus T-Ag is transiently located in the cytoplasm at the same point of the cell cycle (G₁) when p193-induced cell death occurs.

Loss of p193 Activity Promotes Proliferation

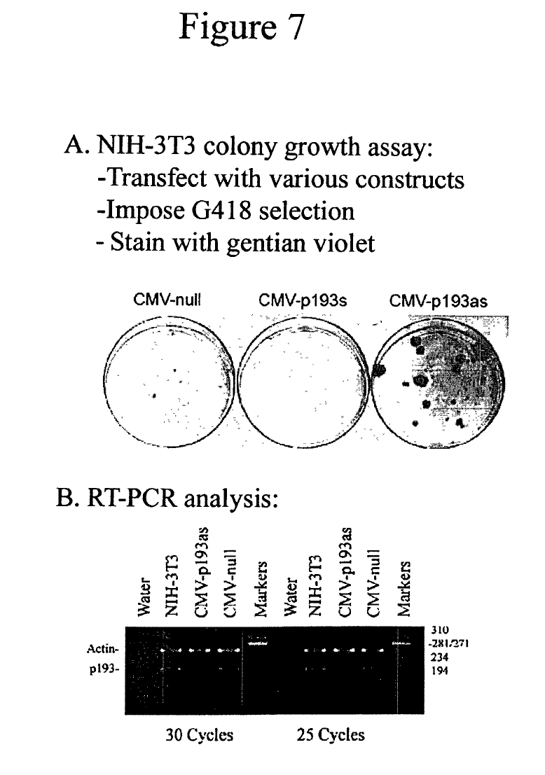

The data presented above indicate that forced expression of p193 promotes apoptosis prior to the onset of S-phase. A colony growth assay employing a p193 antisense construct (CMV-p193as) was used to determine the consequences of diminished p193 expression. Transfection of NIH-3T3 cells with CMV-p193as resulted in markedly increased colony size as compared to transfection with CMV-null (a control expression vector lacking insert, see FIG. 7A). Northern blots of parallel cultures indicated that p193 transcripts were markedly diminished in cultures transfected with the anti-sense construct (data not shown). As expected, transfection with CMV-193s (an expression vector encoding p193 in the sense orientation) yielded no visible colonies (FIG. 7A), consistent with the pro apoptotic activity of p193 noted above.

To confirm that expression of the antisense construct effected levels of the endogenous p193 transcript, RT-PCR analyses were performed (see FIG. 7B). The amplification primers were selected to permit co-amplification of a fragment of the beta-actin transcript and a fragment of the p193 transcript present in the endogenous gene but not in the expression vector. The relative ratio of the beta-actin amplification products and the p193 amplification products provides a quantitative assessment steady-state levels of endogenous p193 transcripts. RNA was prepared from cells expressing the CMV-null vector, from cells expressing the CMV-p193as vector, or from non-transfected NIH-3T3 cells. The relative level of the p193 amplification products are reduced in RNA from cells expressing the CMV-p193as construct vs. the control cells, indicating that the antisense intervention was successful at decreasing steady state levels of the endogenous transcripts (FIG. 7B). Note however that p193 expression was not completely blocked.

### Examples 1-3: Discussion

We have shown that p193, a T-Ag binding protein present in the AT-2 cardiomyocyte tumor cell line, is a new member of the BH3 only pro-apoptosis gene family. Like other BH3 only proteins, p193-induced apoptosis can be antagonized by co-expression of pro-survival Cl-2 family members (in our case, Bcl-X₁ was tested). p193 differs markedly in size as compared to other BH3 only family members; the next largest family member, BID, is only 21.95 kd (Wang, K., Yin, X. M., Chao, D. T., Milliman, C. L., and Korsmeyer, S. J. (1996) Genes Dev. 10, 2859-2869). Co-expression of T-Ag antagonizes p193-induced apoptosis in transiently transfected cells, and results in the cytoplasmic sequestration of both proteins. Moreover, T-Ag is localized in the cytoplasm of AT-2 cardiomyocytes during G1, the same point of the cell cycle where p193 induces apoptosis. These data are consistent with the notion that T-Ag/p193 binding in the cytoplasm may modify or abrogate p193 activity in SV40 transformed cells.

If p193 binding is important for T-Ag mediated transformation, we would anticipate that mutations at and/or near the p193 binding site would diminish T-Ag transforming activity. Indeed, previous mutational analyses have identified transformation activity at the N-terminus of T-Ag. For example, Kohrman and Imperiale (Kohrman, D. C. and Imperiale, M. J. (1992) J. Virol. 66, 1752-1760) demonstrated that amino acid residues 1-108 were required to effectively transform B2-1 cells, and that a ca. 185 kd protein bound to this region of T-Ag. Moreover, binding between T-Ag and the 185 kd protein was not disrupted by point mutations abrogating the binding of RB family members. Given the similarity in molecular weight and binding specificity, p193 may be the same protein as p185.

Other studies have demonstrated that mutations at T-Ag amino acid residues 1-82 (Marsilio, E., Cheng, S. H., Schaffhausen, B., Paucha, E., and Livingston, D. M. (199) J. Virol. 65, 5647-5652), 3-35 (Zhu, J., Rice, P. W., Gorsch, L., Abate, M., and Cole, C. N. (1992) J. Virol. 66, 2780-2791), and 17-27 (Srinivasan, A., McClellan, A. J., Vartikar, J., Marks, I., Cantalupo, P., Li, Y., Whyte, P., Rundell, K., Brodsky, J. L., and Pipas, J. M. (1997) Mol. Cell. Biol, 17, 4761-4773) all impact upon transforming activity in selected cell types. Some of these mutants are thought to disrupt the N-terminal J domain, a sequence motif which functions as a Dnaj molecular chaperone (Srinivasan, A., McClellan, A. J., Vartikar, J., Marks, I., Cantalupo, P., Li, Y., Whyte, P., Rundell, K., Brodsky, J. L., and Pipas, J. M. (1997) Mol. Cell. Biol. 17, 4761-4773; Campbell, K. S., Mullane, K. P., Aksoy, I. A., Stubdal, H., Zalvide, J., Pipas, J. M., Silver, P. A., Roberts, T. M., Schaffhausen, B. S., and DeCaprio, J. A. (1997) Genes Dev. 11, 1098-1110). DnaJ binds to members of the 70 kd heat shock protein family, and this complex facilitates correct protein folding, formation of multi-protein complexes, and protein transport across intracellular membranes (Gething, M. J. and Sambrook, J. (1992) Nature 355, 33-45). Although our data indicate that the C-terminal boundary of the p193 binding site resides between T-Ag amino acids 92 through 147, the N-terminal boundary of the binding site is not yet mapped. Mutations encompassing residues upstream of amino acid 92 could alter p193/T-Ag binding, by direct disruption to the binding domain or by altering the tertiary structure of T-Ag. Confirmation of the important of p193 binding for T-Ag transforming activity requires precise mapping of the binding site followed by assessment of transforming activity with appropriately mutated T-Ag expression constructs. Finally, given the relative proximity of the p193 and RB family member binding sites, it will be of interest to determine if RB, p107 and/or p193 sterically compete for T-Ag binding. Such a mechanism could account for the absence of RB in anti-T-Ag immune precipitates from the myocardial cell lines, despite the presence of hypophosphorylated RB in total protein prepared from these cells (FIG. 1, see also Kim, K. K, Soonpa, M. H., Daud, A. I., Koh, G. Y., Kim, J. S., and Field, L. J. (1994) J. Biol. Chem. 269, 22607-22613).

p193 also appears to be unique among the BH3 only family members with respect to its ability to bind to T-Ag. However it is of interest to note that the BH3 only proteins Bik and BNIP-3 (as well as Bax and Bak, pro-apoptosis proteins containing BH1, BH2 and BH3 domains) are able to bind to adenoviral E1B 19K protein (Farrow, S. N., White, J. H., Martinou, L, Raven, T., Pun, K. T., Grinham, C. J., Martinou, J. C., and Brown, R. (1995) Nature 374, 731-733; Boyd, J. M., Malstrom, S., Subramanian, T., Venkatesh, L. K., Schaeper, U., Elangovan, B., D'Sa-Eipper, C., and Chinnadurai, G. (1994) Cell 79, 341-351; Boyd, J. M., Gallo, G. J., Elangovan, B., Houghton, A. B., Malstrom, S., Avery, B. J., Ebb, R. G., Subramanian, T., Chittenden, T., Lutz, R. J., and et al. (1995) Oncogene 11, 1921-1928; Han, J., Sabbatini, P., Perez, D. Rao, L., Modha, D., and White, E. (1996) Genes Dev. 10, 461-477). It is thought that the anti-apoptotic activity of the E1B 19K protein is due at least in part to binding with pro-apoptosis Bcl-2 family members (White, E. (1995) Current Topics in Micro. & Immuno. 34-58). Previous studies have identified a T-Ag anti-apoptotic activity at amino acid residues 525 through 541 which appeared to act independently of p53 sequestration (Conzen, S. D., Snay, C. A., and Cole, C. N. (1997) J. Virol. 71, 4536-4543). These authors noted a significant degree of sequence homology between this region of T-Ag and amino acid residues 77 through 93 in E1B 19K as well as Bcl-2 amino acid residues 133 through 151. Although these observations suggest that the binding activity at T-Ag amino acid residues 525-542 might be functionally similar to E1B 19K protein sequestration of pro-apoptosis proteins, experiments aimed at establishing direct binding of T-Ag to Bak were unsuccessful (Conzen, S. D., Snay, C. A., and Cole, C. N. (1997) J. Virol. 71, 4536-4543).

The results from the antisense transfection experiment indicated that loss of p193 activity is associated with marked growth enhancement in NIH-3T3 cells. The increase in growth rate is in excess to that which we would anticipate from simple inhibition of apoptosis in the NIH-3T3 cells, which occurs somewhat infrequently under the growth conditions employed. In support of this, preliminary experiments have shown that cells expressing the CMV-p193as construct exhibit higher DNA synthesis labeling indices as compared to cells expressing control constructs (Tsai, unpublished results). This observation is consistent with the notion that p193 may function at a cell cycle checkpoint, and that transit through the checkpoint is accelerated in the absence of p193 activity. This hypothesis is supported in part by the serum starvation experiment described above, which indicated that at least a limited degree of cell cycle progression is required for actuation of the p193-mediated cell death program. Thus p193 is only able to trigger cell death after transit through a specific point in G₁ (i.e. the presumed cell cycle check point), and accumulation of the protein in itself is not harmful to the cell. The observation that cytoplasmic T-Ag localization occurs during the same point of the cell cycle lends additional credence to this notion. Further insight into the molecular pathway of p193 must await the generation of additional loss of function models.

Our efforts to characterize p193 were motivated in part by the hope of identifying potential therapeutic targets with which to engender regenerative growth in diseased hearts. In this regard it is of interest to note that transfection of primary cardiomyocyte cultures with E1A or E2F-1 results in a prompt apoptotic response which is only partially abated by co-expression of E1B, Bcl-2 or abrogation of p53 activity (Kirshenbaum, L. A. and Schneider, M. D. (1995) J. Biol. Chem. 270, 7791-7794; Kirshenbaum, L. A., Abdellatif, M., Chakraborty, S., and Schneider, M. D. (1996) Dev. Biol. 179, 402-411; Liu, Y. and Kitsis, R. N. (1996) J. Cell Biol. 133, 325-334; Agah, R., Kirshenbaum, L. A., Abdellatif, M., Truong, L. D., Chakraborty, S., Michael, L. H., and Schneider M. D. (1997) J. Clin. Invest. 100, 2722-2728; Bishopric, N. H., Zeng, G. Q., Sato, B., and Webster, K. A. (1997) J. Biol. Chem. 272, 20584-20594). In contrast, transformation with T-Ag results in sustained cardiomyocyte proliferation. This observation suggests that, in cardiomyocytes, T-Ag possesses an anti-apoptotic activity which is lacking in E1A and E2F-1. Given that p193 is a pro-apoptotic T-Ag binding protein, and that T-Ag expression does not elicit an apoptotic response in cardiomyocytes, it will be of interest to determine if abrogation of p193 activity can antagonize E1A and/or E2F-1 induced cardiomyocyte apoptosis.

Abrogation of p193 activity may also have a cardioprotective effect under pathophysiological conditions which promote cardiomyocyte apoptosis. Numerous descriptive studies have established the presence of apoptotic cardiomyocytes in a variety of cardiovascular diseases including dilated cardiomyopathy, ischemic cardiomyopathy, arrhythmogenic right ventricular dysplasia, acute myocardial infarction, myocarditis, allograft rejection, and preexcitation syndromes (reviewed in Haunstetter, A. and Izumo, S. (1998) Circulation Research 82, 1111-1129). In particular apoptosis and resulting cardiac remodeling may contribute to the onset of dilated cardiomyopathy and heart failure (reviewed in Anversa, P., Leri, A., Beltrami, C. A., Guerra, S., and Kajstura, J. (1998) Lab. Invest. 78, 767-786). Studies in transgenic mice have implicated a number of signal transduction pathways, including the IL-6 cytokine family/gp130/LIF receptor (Hirota, H., Chen, J., Betz, U. A., Rajewsky, K, Gu, Y., Ross, J., Jr., Muller, W., and Chien, K. R. (1999) Cell 97, 189-98), the TNF-β/TNFR1 (Kubota, T., McTiernan, C. F., Frye, C. S., Slawson, S. E., Lemster, B. H., Koretsky, A. P., Demetris, A. J., and Feldman, A. M. (1997) Circulation Research 81, 627-635; Bryant, D., Becker, L., Richardson, J., Shelton, J., Franco, F., Peshock, R., Thompson, M., and Giroir, B. (1998) Circulation 97, 1375-1381), catecholamine/Gsalpha (Geng, Y. J., Ishikawa, Y., Vatner, D. E., Wagner, T. E., Bishop, S. P., Vatner, S. F., and Homcy, C. J. (1999) Circulation Research 84, 34-42), and cAMP/CREB (Fentzke, R. C., Korcarz, C. E., Lang, R. M., Lin, H., and Leiden, J. M. (1998) J. Clin. Invest. 101, 2415-2426) cascades. The role to which p193 may participate in these processes remains to be established.

In summary, the data presented here indicate that p193 is a new member of the BH3 only pro-apoptosis gene family. p193 promotes cell death during G₁, prior to the onset of DNA synthesis. T-Ag is localized in the cytoplasm during the same phase of the cell cycle, and co-expression of T-Ag antagonizes p193-induced cell death and results in the cytoplasmic localization of both proteins. p193 binds to the N-terminus of T-Ag in a region which contributes to transforming activity in some cell types. Collectively, these results suggest that T-Ag possesses an anti-apoptosis activity, independent of p53 sequestration, which is actuated by T-Ag/p193 binding in the cytoplasm.

### Example 4

### Characterization of a Dominant-negative p193 Mutation

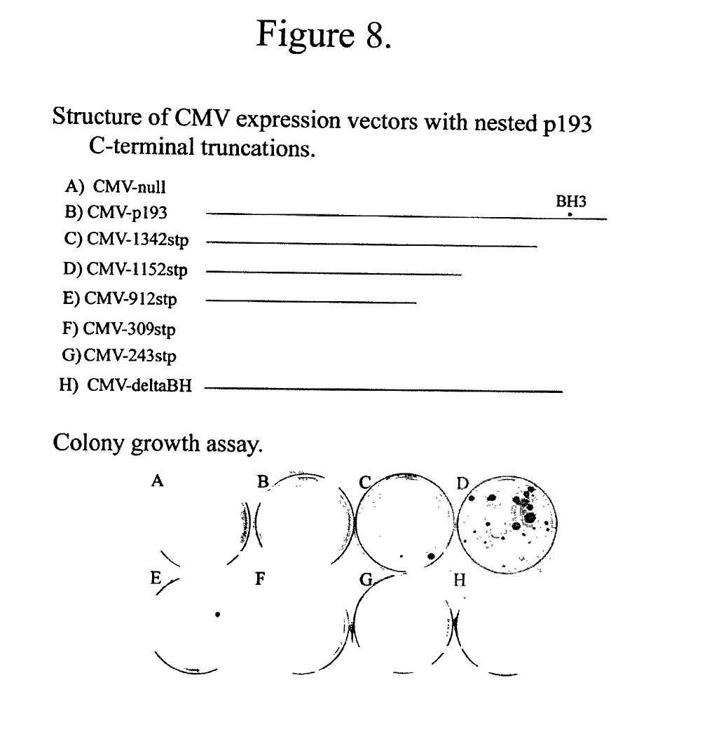

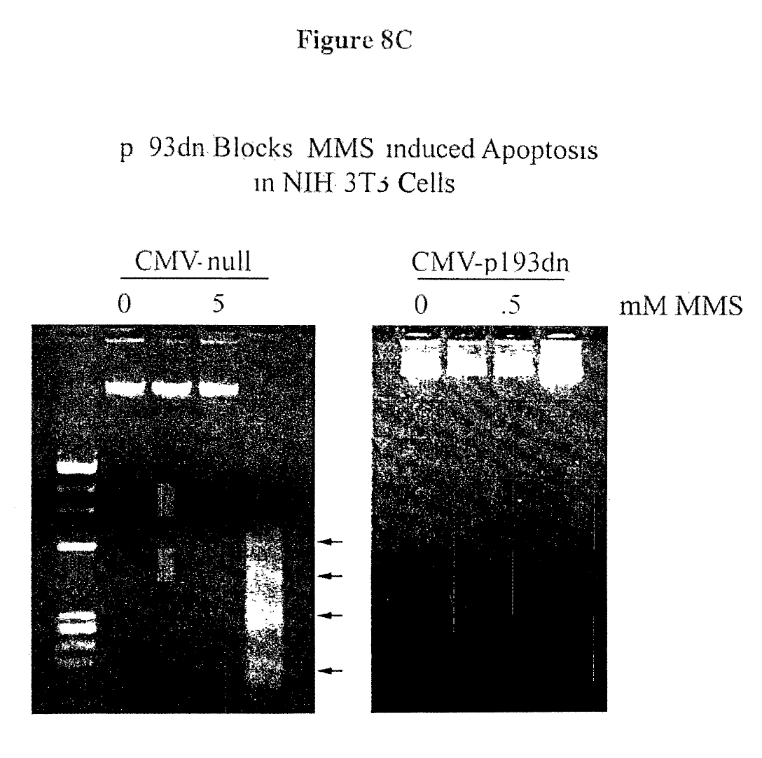

Colony growth assay in NIH-3T3 cells indicate that decreased p193 activity as a consequence of anti-sense expression results in increased rates of cell growth (FIG. 7). A priori, expression of dominant negative variants of p193 should also result in increased rates of cell growth. We generated a nested series of p193 cDNAs harboring progressively greater C-terminal truncations. The cDNAs were subcloned into a CMV expression vector. The structure of the p193 variants are depicted in FIG. 8A. The expression vectors also carried a neomycin-resistance cassette. NIH-3T3 cells were transfected with the various expression vectors, and the cells were cultured in the presence of G418. After 15 days of selection the cultures were fixed and stained with gentian violet. Representative cultures of cells transfected with the various constructs are shown in FIG. 8B. Cells transfected with the CMV-null vector represent the negative control (this reflects the rate of growth in the absence of any positive or negative cell cycle regulators, see culture plate A). Consistent with the pro-apoptotic activity of p193 no colonies were observed in cultures transfected with full-length p193 (amino acid residues 1-1689; culture plate B). A slight enhancement in cell growth was detected in cells transfected with a vector expressing p193 amino acid residues 1 through 1342 (culture plate C). Marked growth enhancement was observed in cells transfected with a vector expressing p193 amino acid residues 1 through 1152 (culture plate D). Little or no growth enhancement was observed in cells transfected vectors expressing p193 molecules 1-912, 1-309, and 1-243 (culture plates E-G, respectively), or with a vector expressing a p193 molecule where only the BH3 domain was deleted (culture plate F). These data indicate that expression of p193 amino acid residues 1-1152 promotes growth in NIH-3T3 cells, a trend which is also observed with expression of p193 anti-sense constructs (see FIG. 7). Based on this, sequences encoded by p193 amino acid residues 1-1152 have been designated “p193dn”, for p193 dominant negative. The greater effect of the p193dn on cells growth as compared to the p193 antisense constructs likely reflects the fact that expression of the antisense construct does not completely eliminate endogenous p193 transcripts (FIG. 7).

The above-described experiments provide a preliminary characterization of sequence modifications which bestow a dominant negative phenotype on p193 (as evidenced by the property of bestowing enhanced growth and anti-apoptotic activity in NIH-3T3 cells, and blocking apoptosis in cardiomyocytes). Further delineations of the p193 amino acid residues responsible for these characteristics is easily accomplished by one skilled in the art. For example, fine scale deletions encompassing the regions defined in the experiments described above would further delineate the amino acids required to bestow the dominant negative phenotype. The use of amino acid substitutions which retain gross protein structure but inhibit specific amino acid interactions are readily performed with generic molecular biology techniques. The NIH-3T3, ES-derived cardiomyocyte growth assays and targeted cardiac expression in transgenic mice, as described in other sections of this patent application, provide the requisite experimental endpoints with which to characterize the modified p193 constructs.

To further confirm that p193dn encodes dominant negative activity, we tested its ability to block apoptosis in response to treatment with methyl methanesulfonate (MMS). NIH-3T3 cells were transfected with a CMV-null expression construct, or a CMV-p193dn expression construct, and stable cell lines were generated. The cells were then incubated in growth medium supplemented with MMS (0 mM, 0.1 mM, 0.5 mM or 1 mM) for 3 hrs. at 37° C. Cells were then harvested and apoptosis was measured by determining the degree of DNA fragmentation (nucleosomal cleavage of DNA is diagnostic for apoptosis). Extensive fragmentation is apparent in DNA prepared from the CMV-null control cells cultured in the presence of 1 mM MMS; in contrast no DNA fragmentation is apparent DNA prepared from CMV-p193dn cells following lmM MMS treatment (see FIG. 8C). These data indicate that the p193dn construct blocks MMS-induced apoptosis in NIH-3T3 cells, and supports the notion that this variant encodes dominant negative activity.

### Example 5

### Generation of Transgenic Mice Expressing p193dn in the Heart

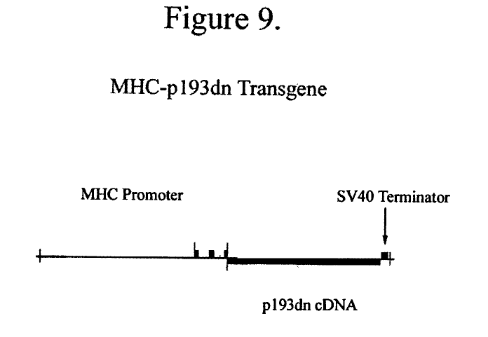

Transgenic mice were generated to examine the potential cardioprotective effects of p193dn on the heart. Cardiac expression was targeted using the α-cardiac myosin heavy chain (MHC) promoter. The MHC promoter consisted of 4.5 kb of 5′ flanking sequence and 1 kb of the gene encompassing exons 1 through 3 up to but not including the initiation codon (Gulick, J., A. Subramaniam, J. Neumann, and J. Robbins (1991) Isolation and characterization of the mouse cardiac myosin heavy chain genes. Journal of Biological Chemistry 266:9180-9185). A cDNA encoding p193dn was inserted downstream of the promoter, followed by the SV40 early region transcription terminator (SV40 nucleotide residues #2586-2452, see Reddy, V. B., B. Thimmappaya, R. Dhar, K. N. Subramanian, B. S. Zain, J. Pan, P. K. Ghosh, M. L. Celma, and S. M. Weissman (1978) The genome of simian virus 40. Science 200:494-502.). The resulting transgene was designated MHC-p193dn. A schematic diagram of the transgene is presented in FIG. 9. To generate transgenic mice, the transgene DNA was digested with restriction enzymes to separate the MHC-p193 sequences from the vector, and the insert purified from an agarose gel using Geneclean glass beads (Bio 101, Vista Calif.). Purified insert DNA was microinjected into inbred C3HeB/FeJ (Jackson Laboratories, Bar Harbor Mass.) zygotes using standard methodologies [3]. The microinjected embryos were cultured in vitro to the two cell stage, and then implanted into pseudopregnant SW/Taconic (Taconic Farms, Germantown N.Y.) female mice. For all surgeries, mice were anesthetized with 2.5% Avertin (0.015 ml/g bodyweight IP, Fluka Biochemicals, Ronkomkoma, N.Y.). All manipulations were performed according to NIH and Institutional Animal Care and Use Guidelines.

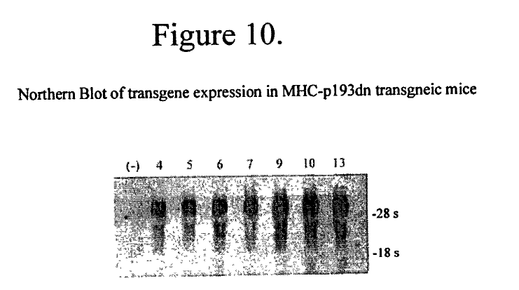

Pups derived from the microinjected embryos were screened for the presence of the transgene using diagnostic PCR amplification as described (Steinhelper, M. E., K. L. Cochrane, and L. J. Field (1990) Hypotension in transgenic mice expressing atrial natriuretic factor fusion genes. Hypertension 16:301-307). 13 transgenic founder animals were obtained from the embryos microinjected with the MHC-p193dn construct. Seven of the founders were randomly selected to establish transgenic lineages. Adult heart RNA prepared from F1 transgenic animals was used to stratify the levels of p193dn expression between the different lines. High levels of p193dn transcripts were observed in all of the lines (designated MHC-p193dn line 4, 5, 6, 7, 9, 10 and 13, see FIG. 10): based on these analyses line 13 was selected for additional experiments.

### Example 6

### Demonstration that Expression of p193dn is Cardioprotective in Vivo

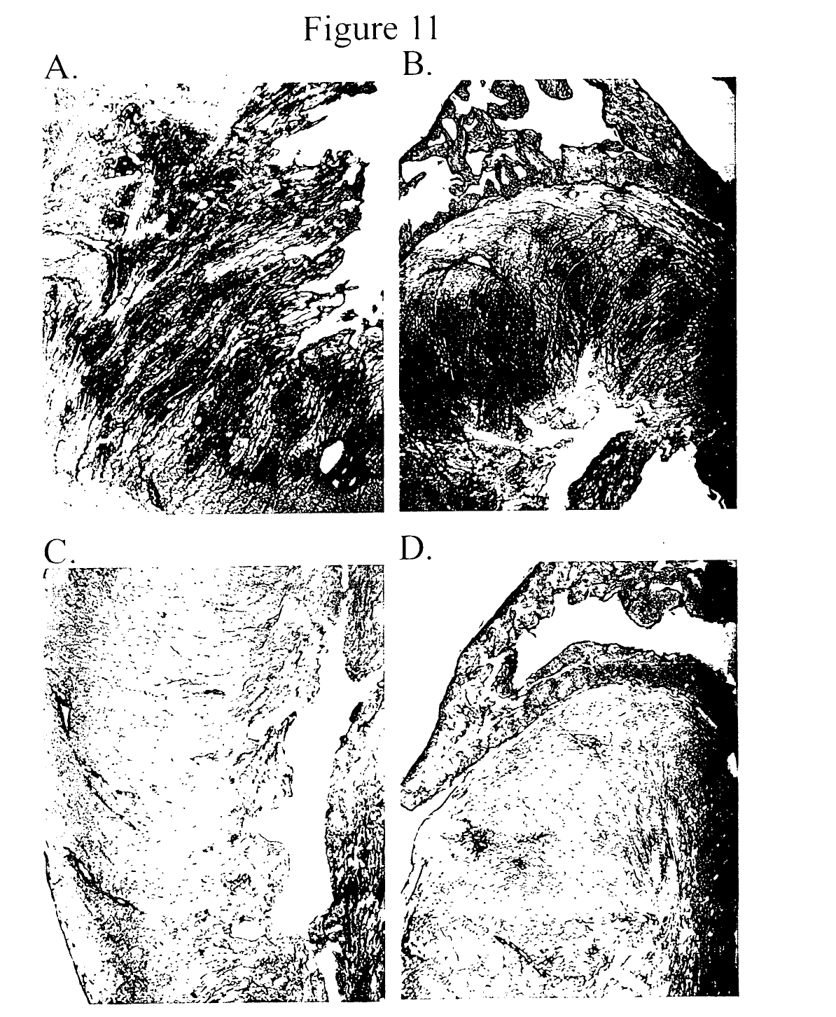

Myocardial damage in response to chronic isoproterenol infusion was monitored in control and MHC-p193dn transgenic mice to determine if transgene expression was cardioprotective. Non-transgenic control and MHC-p193dn transgenic mice were identified and sequestered until they reached 11 weeks of age. Continuous isoproterenol infusion was administered using implanted osmotic mini-pumps (model 2001, Alzet, Palo Alto Calif., flow rate of 1 μl/hr) filled with 0.028 g/ml isoproterenol (dissolved in saline). After seven days of treatment the mice were sacrificed, the hearts harvested, cryoprotected and sectioned using standard histologic techniques (Bullock, G. R. and P. Petrusz (1982) Techniques in immunocytochemistry, Academic Press, London; New York.). Heart sections were then stained with Sirius red (which reacts with collagen to produce a dark signal in the images presented) and counter-stained with fast green (which reacts with muscle cells to produce a light signal in the images presented). The results are shown in FIG. 11. Panels A and B depict sections of a nontransgenic heart after seven days of isoproterenol infusion. Abundant Sirius red staining is apparent throughout the ventricular myocardium (panel A shows the left ventricular myocardium near the apex of the heart, panel B shows the ventricle myocardium near the base of the). The dark staining is indicative of extensive fibrosis which resulted from isoproterenol-induced cardiomyocyte death. Panel C and D depict similar analysis of a MHC-193dn transgenic heart after seven days of isoproterenol infusion. Essentially no dark staining is detected, indicating the absence of fibrosis in the isoproterenol-treated transgenic hearts. This result indicates that expression of the p193dn transgene protects the myocardium from isoproterenol-induced fibrosis. Other studies (Communal C; Singh K; Pimentel D R; Colucci W S (1998) Norepinephrine stimulates apoptosis in adult rat ventricular myocytes by activation of the beta-adrenergic pathway, Circulation 29:98(13):1329-34) have shown that isoproterenol treatment induces cardiomyocyte apoptosis: thus p193dn expression blocks cardiomyocyte apoptosis and the ensuing fibrosis.

### Example 7

### Demonstration that Co-expression of p193dn and p53dn Blocks E1A Induced Apoptosis and Promotes Proliferation in ES Derived Cardiomyocytes

Previous studies have shown that expression of the Adenoviral E1A oncoprotein can reactivate cell cycle ion cardiomyocytes, but this reactivation is immediately followed by apoptotic cardiomyocyte death (Kirshenbaum, L. A. and M. D. Schneider. Adenovirus E1A represses cardiac gene transcription and reactivates DNA synthesis in ventricular myocytes, via alternative pocket protein-and p300-binding domains, J. Biol. Chem. (1995) 270: 7791-7794). Moreover, blocking the p53-regulated apoptotic pathway only partially rescues the cardiomyocytes. A study was therefore employed to determine if co-expression of p193dn and p53dn can block E1A-induced cardiomyocyte apoptosis. The experiment utilized a previously described technique to generate enriched cardiomyocyte cultures from differentiating ES cells (U.S. Pat. Nos. 5,602,301 and 5,733,727 to Field et al.; and Klug, M. G., M. H. Soonpaa, G. Y. Koh, and L. J. Field (1996) Genetically selected cardiomyocytes from differentiating embronic stem cells form stable intracardiac grafts, J. Clin. Invest, 98: 216-224). Undifferentiated ES cells were transfected with an MHC-neor/pGK-hygror transgene alone or in combination with a MHC-E1A, MHC-p193dn and/or MHC-p53dn transgenes. Transfected undifferentiated ES cells were then selected on the basis of hygromycin resistance.

When the hygromycin-resistant clones were sufficiently amplified, the cultures were induced to differentiate. Once cardiomyocytes were apparent in the culture (as evidenced by the presence of beating cells, which usually occurs at 8 days post-induction), the cultures were subjected to G418 selection. Since the neor cassette is under the regulation of the cardiac MHC promoter, only cardiomyocytes survive this selection procedure. After 60 days of G418 selection, the cultures were fixed and stained with PAS to permit visualization of the cardiomyocytes.

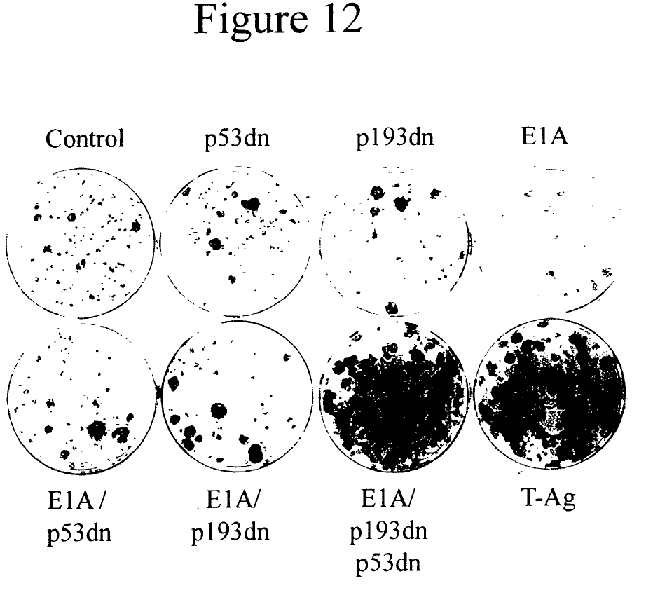

Control plates (transfected with the MHC-neor/pGK-hygror transgene alone) gave rise to numerous colonies of beating myocytes (see the control plate, FIG. 12). This is indicative of the normal rate of ES-derived cardiomyocyte growth in the absence of positive or negative factors. Control dishes transfected with p193dn and p53dn alone are also shown: expression of these genes resulted in a slight increase in cardiomyocyte yield, consistent with their anti-apoptotic activities. In contrast, very few cardiomyocytes were observed in the plate transfected with the MHC-E1A transgene, consistent with previous studies (Kirshenbaum, L. A. and M. D. Schneider. Adenovirus E1A represses cardiac gene transcription and reactivates DNA synthesis in ventricular myocytes, via alternative pocket protein- and p300-binding domains. J. Biol. Chem. 270: 7791-7794, 1995). Co-transfection of E1A and p53dn or E1A and p193dn did not result in a marked increase in cardiomyocyte viability.

In marked contrast to these results, transfection with MHC-E1A, MHC-p53dn and MHC-p193dn gave rise to numerous and substantively larger colonies of cardiomyocytes. Cardiomyocyte colony size was much greater than that observed for the control plates. This result indicates that the combinatorial effect of p53dn and p193dn effectively and completely blocks E1A-induced apoptosis. Moreover, the increase in colony size indicates enhanced proliferation in the ES-derived cardiomyocytes expressing all three transgenes. Thus, co-expression of p193dn and p53dn blocks E1A induced apoptosis and in so doing permits E1A-induced cell cycle activation in ES derived cardiomyocytes.

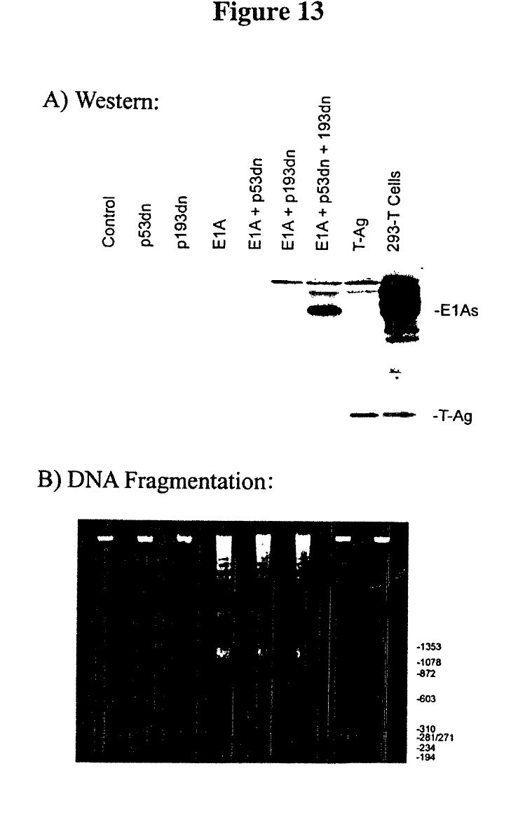

To further characterize the cardiomyocytes, protein was prepared from representative dishes from each of the transfections depicted in FIG. 12. The protein was then subjected to Western blot analysis with anti-E1A or anti-T-Ag antibodies using standard protocols (FIG. 13A). Importantly, no E1A protein was detected in cells expressing E1A alone, or E1A+p53dn, or E1A+p193. In contrast, abundant levels of E1A were detected in cells expressing E1A+P53dn+p193dn. This suggests that E1A is lethal in cardiomyocytes unless both the p53 and p193 pathways are blocked. Moreover, these data indicate that the cardiomyocytes on the E1A alone, or E1A+p53dn, or E1A+p193 culture dishes probably arose from progenitors which did not take up (or alternatively did not express) the E1A construct. The experiment was performed on 60 day old cultures.

To confirm that E1A expression in the absence of co-expression of both p13dn and p193dn induced apoptosis, DNA prepared from 13 day old cultures was analyzed for the degree of DNA fragmentation (nucleosomal cleavage of DNA is diagnostic for apoptosis) (FIG. 13B). Extensive fragmentation is apparent in DNA prepared from cells-expressing E1A alone, or E1A+p53dn, or E1A+p193. In contrast, no fragtmentation was observed in cells expressing E1A+P53dn+p193dn. These data confirm that co-expression of p53dn and p193dn blocks E1A-induced apoptosis in cardiomyocytes.

### Example 8

### Demonstration that p193 is Expressed in a Cell-cycle Dependent Fashion

Many cell cycle regulatory proteins are expressed and/or active during discrete phases of the cell cycle. To determine if this is the case for p193, anti-p193 monoclonal antibodies were produced. A recombinant protein encoding p193 amino acid residues 1153-1689 was used as the immunogen, and monoclonal antibodies were raised, screened and validated using standard approaches. To monitor p193 expression during the cell cycle, NIH-3T3 cells were synchronized by two rounds of serum depletion (starvation media contained 0.1% FBS in DMEM). To monitor cell synchronization, some of the cells were incubated in media containing 3H-thymidine (26 Ci/mmol, Amersham, Buckinghamshire, England) and 10% FBS in DMEM; the cells were processed for autoradiography at various points thereafter to monitor DNA synthesis. The preponderance of non-transfected cells on the same chamber slide reinitiated DNA synthesis by 14 hrs. post-serum replenishment (FIG. 14A), thus establishing the fidelity of the synchronization protocol. Protein prepared at similar time points from parallel dishes was used for Western blot analysis (FIG. 14B). No p193 expression was detected at 2, 4, or 6 hours following the addition of serum. Prominent p193 expression was detected at 8, 10 and 12 hours post serum addition, roughly concomitant with the onset of DNA synthesis. The levels of p193 were markedly reduced in subsequent time points. These data indicate that p193 expression is tightly regulated during cell cycle progression, and that peak levels occur at the G1/S boundary. Interestingly, this is the precise point of the cell cycle where forced expression of p193 induces apoptosis, and is also a point in the cell cycle where T-Ag is localized in the cytoplasm.

### Example 9

### Data Suggesting that Blockage of p193 and p53 Activity can Result in a Proliferative Response to Hypertrophic Stimuli

Many forms of cardiac injury result in an initial phase of hypertrophic growth which compensates for the loss of functional myocytes. Over time, the hypertrophic heart can decompensate, a process which leads to cardiac dilation and ultimately heart failure. Cardiomyocyte apoptosis is frequently observed during this process. It is also well established a large number of gene products normally associated with cell proliferation are induced during cardiac hypertrophy (see for example Izumo, S. et al Proc. Natl. Acad. Sci. USA 85, 339-343; Mulvagh, S. L. et al., Biochem. Biophys. Res. Commun. 147, 627-636; Simpson, P. C. Annual Review of Physiology, 51, 189-202). It is possible that hypertrophic stimuli are in fact mitogenic stimuli, and that in the mature cardiac myocyte the response to such stimuli is to first increase cell size, and then transit G1/S. Our data clearly indicate that two pro-apoptotic pathways (the p53 and p193 pathways) are activated in cardiomyocytes which are experimentally induced to proliferate. The apoptotic response observed during the process of decompensation might result from the initiation of cell cycle activity in the presence of active p53 and p193 pathways.

It follows then that if the p193 and p53 pathways are blocked, hypertrophic stimuli might result in direct cell cycle activity. To test this, undifferentiated ES cells were transfected with the MHC-neor/pGK-hygror transgene in combination with both the MHC-p193dn and MHC-p53dn transgenes. Transfected cells were enriched by virtue of their resistance to hygromycin, and then induced to differentiate. The ES-derived cardiomyocytes were then enriched by virtue of their resistance to G418. Cardiomyocyte growth was then compared in these cultures in the presence vs. absence of exogenous isoproterenol (1 μM mg/ml) for 58 days. Markedly enhanced cardiomyocyte colony size was apparent in the isoproterenol treated cultures, consistent with the presence of increase cell numbers (see FIG. 16). This suggests that relaxation of cell cycle apoptotic check-points renders cardiomyocytes proliferative to what would otherwise be a hypertrophic stimuli.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

All publications cited herein are indicative of the level of skill in the art and are hereby incorporated by reference as if each had been individually incorporated by reference and fully set forth.

What is claimed is:

1. A method for modifying the cell cycle of a cell, comprising modifying the level of p193 protein in the cell and/or interfering with the p193 signal transduction pathway in the cell.

2. The method of claim 1, which comprises decreasing the level of pro-apoptotic p193 protein in the cell, so as to suppress apoptosis in and/or increase the proliferative potential of the cell.

3. The method of claim 1, which comprises increasing the level of pro-apoptotic p193 protein in the cell, so as to induce apoptosis in the cell.

4. The method of any of claims 1-3, wherein the cell is a mammalian cell.

5. The method of claim 4, wherein the cell is a human cell.

6. The method of claim 2, which comprises introducing nucleic acid encoding a portion of or all of the p193 protein into the cell in the antisense orientation, so as to decrease the level of p193 protein activity in the cell.

7. The method of claim 1, which comprises introducing nucleic acid encoding a dominant-negative p193 protein into the cell, so as to suppress apoptosis and/or increase the proliferative potential of the cell.

8. The method of claim 3, which comprises introducing nucleic acid encoding a pro-apoptotic p193 protein into the cell, so as to express said p193 protein and induce apoptosis in the cell.

9. The method of claim 1, also comprising modifying the level of p53 protein in the cell and/or interfering with the p53 signal transduction pathway into the cell.

10. The method of claim 1 or 9, also comprising modifying the level of E1A protein in the cell.

11. An expression vector including nucleic acid encoding a p193 protein.

12. The expression vector of claim 11 wherein said nucleic acid is in the antisense orientation.

13. The expression vector of claim 11 wherein said p193 protein is a pro-apopotic p193 protein.

14. The expression vector of claim 11 wherein said p193 protein includes a dominant negative mutation.

15. A host cell comprising introduced nucleic acid encoding a p193 protein.

16. The host cell of claim 15 wherein said nucleic acid encodes a pro-apoptotic p193 protein.

17. The host cell of claim 15 wherein said nucleic acid encodes a p193 protein including a dominant negative mutation.

18. An isolated p193 protein.

19. The isolated p193 protein of claim 18, having the amino acid sequence set forth in SEQ ID NO: 2 or in SEQ. ID NO: 4.

20. A composition comprising an isolated p193 protein of claim 18 or 19, and a carrier.

21. A method of inducing apoptosis in a cell, comprising expressing in said cell an amount of a pro-apoptotic p193 protein effective to induce apoptosis in said cell.

22. The method of claim 21 wherein said cell is an inappropriately proliferative cell.

23. An expression vector comprising a nucleic acid sequence encoding the amino acid sequence of SEQ ID NO: 2 or SEQ ID NO: 4 or an amino acid sequence having at least about 70% identity to the amino acid sequence of SEQ ID NO: 2 or SEQ ID NO: 4.

24. An expression vector comprising a nucleic acid sequence encoding a polypeptide having the amino acid sequence of SEQ ID NO: 2 from residue 1 to residue 1152 or of SEQ ID NO: 4 from residue 1 to 1173, or an amino acid sequence having at least about 70% identity to the amino acid sequence of SEQ ID NO: 2 from residue 1 to residue 1152 or of SEQ D NO: 4 from residue 1 to residue 1173.

25. The expression vector of claim 24, wherein said polypeptide suppresses apoptosis and/or induces proliferation in a cell in which it is expressed.

26. An expression vector comprising a nucleic acid sequence having at least 70% identity to nucleotides 62 to 5128 of SEQ ID NO: 1 or nucleotides 87 to 5183 of SEQ ID NO: 3.

27. An expression vector comprising a nucleic acid sequence having at least about 70% identity to nucleotides 62 to 3517 of SEQ. ID NO: 1 or to nucleotides 87 to 3615 of SEQ. ID NO: 4.

28. A protein of claim 18, said protein being a recombinant protein.

29. A recombinant protein of claim 26, which has the amino acid sequence set forth in SEQ ID NO: 2 or SEQ ID NO: 4 or an amino acid sequence having at least about 70% identity to the amino acid sequence set forth in SEQ ID NO: 2 or SEQ ID NO: 4.

30. A recombinant protein of claim 28, which has the amino acid sequence set forth in SEQ ID NO: 2 from residues 1 to 1152 or set forth in SEQ ID NO: 4 from residues 1 to 1173, or an amino acid sequence having at least about 70% identity to the amino acid sequence set forth in SEQ ID NO: 2 from residues 1 to 1152 or set forth in SEQ ID NO: 4 from residues 1 to 1173.

31. A composition, comprising an antibody to a p193 protein.

32. The composition of claim 31, wherein said antibody is a monoclonal antibody.

33. The composition of claim 31, wherein said antibody is a polyclonal antibody.

34. A method for producing a p193 protein, comprising culturing a host cell having introduced DNA encoding a p193 protein under conditions suitable for expression of said introduced DNA.

35. An isolated apoptosis-associated protein comprising a BH3 domain including the amino acid sequence:
Leu Lys Ala His Gly Asp Glu.

36. An isolated nucleic acid molecule encoding an apoptosis-associated protein comprising a BH3 domain including the amino acid sequence:
Leu Lys Ala His Gly Asp Glu.

37. A method for screening an agent for effect on the cell cycle of a cell, comprising contacting a cell having introduced nucleic acid encoding a p193 protein with the agent and assessing the effect of the agent on the cell.

38. A method of claim 37 wherein the introduced nucleic acid is introduced DNA encoding a pro-apoptotic p193 protein.

39. A method of claim 38, wherein the introduced DNA comprises a nucleic acid sequence encoding the amino acid sequence of SEQ ID NO: 2 or SEQ ID NO: 4 or an amino acid sequence having at least about 70% identity to the amino acid sequence of SEQ ID NO: 2 or SEQ ID NO: 4.

40. A method of claim 39, wherein the introduced DNA comprises a nucleic acid sequence encoding the amino acid sequence of SEQ ID NO: 2 or SEQ ID NO: 4.
